SEQUENCE LISTING (4 sequences)

SEQ ID NO: 1 - protein, 6 aa, synthetic construct
ENLYFQ

SEQ ID NO: 2 - protein, 18 aa, synthetic construct
LKQINVIAGVKEPIRAYG

SEQ ID NO: 3 - protein, 17 aa, synthetic construct
LKQINVIAGVKPIRAYG

SEQ ID NO: 4 - protein, 6 aa, synthetic construct
EXLYZQ

(19) United States
(12) Patent Application Publication
(10) Pub. No.: US 2026/0357338 A1
(43) Pub. Date: Nov. 12, 2026

(54) ENZYMATIC MODIFICATION OF DNA-ENCODED LIBRARIES

(71) Applicant: University of Utah Research Foundation, Salt Lake City, UT (US)

(72) Inventors: Vahe Bandarian, Salt Lake City, UT (US); Karsten A.S. Eastman, Salt Lake City, UT (US)

(21) Appl. No.: 19/651,330

(22) Filed: Apr. 17, 2026

Related U.S. Application Data
(60) Provisional application No. 63/790,919, filed on Apr. 18, 2025.

(51) Int. Cl. C12Q 1/6869; C12Q 1/48; C40B 30/04; C40B 40/08; C40B 70/00

(52) U.S. Cl. CPC C12Q 1/6869; C12Q 1/48; C12Y 504/99; C40B 30/04; C40B 40/08; C40B 70/00

(57) ABSTRACT

The present disclosure is concerned with DNA-encoded libraries (DELs) and enzymatic modification of DNA-encoded libraries. Also described are methods of screening using DNA-encoded libraries. This abstract is intended as a scanning tool for purposes of searching in the particular art and is not intended to be limiting of the present invention.

### CROSS-REFERENCE TO RELATED APPLICATIONS

This Application claims the benefit of U.S. Application No. 63/790,919, filed on Apr. 18, 2025, the contents of which is incorporated herein by reference in its entirety.

### STATEMENT REGARDING FEDERALLY SPONSORED RESEARCH

This invention was made with government support under GM122740 and R35 GM126956 awarded by the National Institutes of Health. The government has certain rights in the invention.

### REFERENCE TO SEQUENCE LISTING

This Sequence Listing submitted Apr. 17, 2026 as a xml file named “21101.0506U2.xml,” created on Apr. 14, 2026, and having a size of 8,192 bytes is hereby incorporated by reference pursuant to 37 C.F.R. § 1.52(e)(5)

### BACKGROUND

Macrocyclic peptides (MPs) refer to cyclic peptidic structures having a ring size of at least twelve atoms (Driggers et al. (2008) Nat. Rev. Drug Discovery 7: 608-624). The molecular weight of MPs can vary between five hundred to several thousand Dalton, which allows them to bridge the molecular worlds of small molecules and macromolecules such as antibodies (Vinogradov et al. (2019) J. Am. Chem. Soc. 141: 4167-4181; Terrett, N. K. (2010) Drug Discovery Today 7: e97-e104). For example, because MPs are constrained with preorganized residues, which results in minimized entropic loss upon binding to targets, they have the potential to yield binding affinities comparable to those of antibodies (Vinogradov et al. (2019) J Am. Chem. Soc. 141: 4167-4181). Moreover, MPs are amenable to structural modification via chemical syntheses such that their pharmacokinetic and pharmacodynamics properties can be manipulated (Fosgerau and Hoffmann (2015) Drug Discovery Today 20: 122-128). Thus, MPs represent a diverse class of compounds for therapeutic research.

One technique to identify new macrocyclic hits is to utilize DNA-encoded chemical libraries (DELs) (Kunig et al. (2018) Biol. Chem. 399: 691-710). DELs are collections of synthetic compounds linked to unique oligonucleotide tags (Neri and Lerner (2018) Annu. Rev. Biochem. 87: 479-502). Similar to phage display, phenotype and genotype are linked and allow for the production of MP libraries. The target is then typically immobilized on a solid support and incubated with the library members (Decurtins et al. (2016) J. Nat. Protoc. 11: 764-780). Non-binding library members are removed by subsequent washes while the target-bound encoded compounds are submitted to PCR amplification, to obtain enough material for high throughput DNA sequencing and hit identification. Id. Unfortunately, existing approaches to generate MPs in the area of DEL are quite limited, enabling formation of a single macrocycle. Further, the encoding DNA often interferes with the cyclization process, leading to low efficiency, reduced structural diversity, and inconsistent product outcomes.

Despite the therapeutic potential of MPs, the ability to identify new MP hits having diverse chemical structures remains limited. Thus, there remains a need in the art for methods of preparing DNA-encoded libraries of MPs having multivalent, nested, and complex polymacrocyclic architectures. These needs and others are addressed herein.

### SUMMARY

In accordance with the purpose(s) of the invention, as embodied and broadly described herein, the invention, in one aspect, relates to deoxyribonucleic acid- (DNA-) encoded libraries (DEL) and enzymatic modification of DNA-encoded libraries. Also disclosed are methods of screening using a DNA-encoded libraries.

Thus, disclosed are deoxyribonucleic acid- (DNA-) encoded libraries comprising a plurality of complexes, wherein each complex comprises: (a) one or more DNA barcodes; and (b) one or more compounds having a structure represented by a formula:

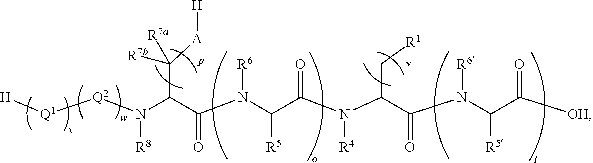

wherein o is 0, 1, 2, 3, 4, 5, 6, 7, 8, or 9; wherein p is 1 or 2; wherein t is an integer from 0 to 500; wherein v is 1, 2, 3, 4, or 5; wherein w is 0 or 1; wherein x is 0 or 1; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

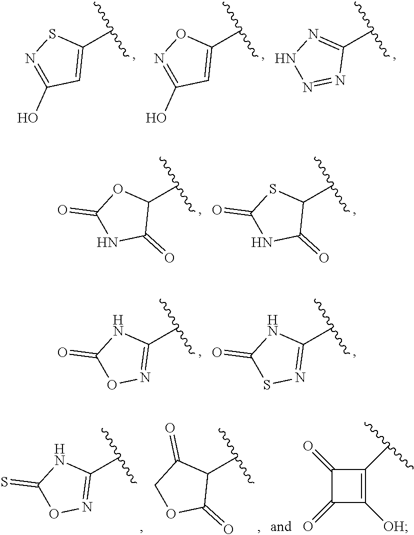

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, wherein at least one of the DNA barcodes is attached to at least one of the compounds.

Also, disclosed are deoxyribonucleic acid- (DNA-) encoded libraries comprising a plurality of complexes, wherein each complex comprises: (a) one or more DNA barcodes; and (b) one or more compounds having a structure represented by a formula:

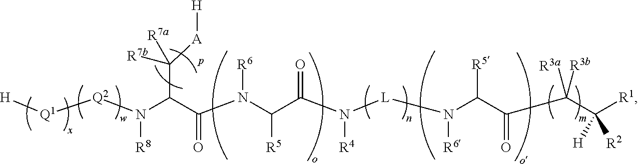

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein x is 0 or 1; wherein A is S or Se; wherein A is S or Se; wherein L, when present, is selected from C2-C4 alkyl, —(C1-C4 alkyl)(OCH₂CH₂)_{q}, and a structure selected from:

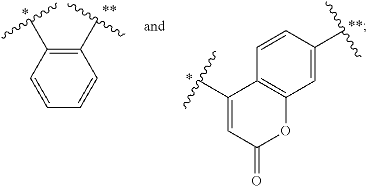

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

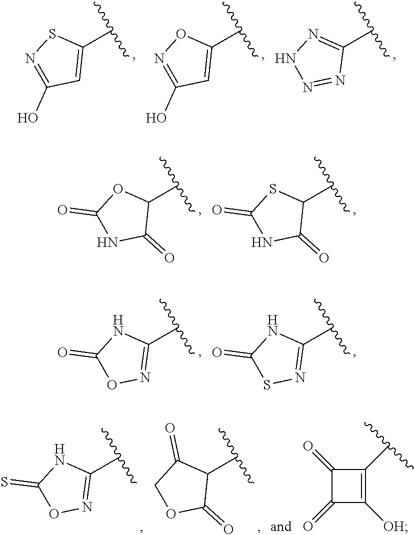

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, wherein at least one of the DNA barcodes is attached to at least one of the compounds.

Also disclosed are deoxyribonucleic acid- (DNA-) encoded libraries comprising a plurality of complexes, wherein each complex comprises: (a) one or more DNA barcodes; and (b) one or more thioether or selenoether compounds having a structure represented by a formula:

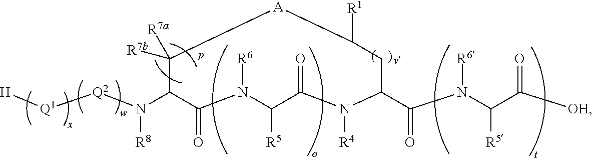

wherein o is 0, 1, 2, 3, 4, 5, 6, 7, 8, or 9; wherein p is 1 or 2; wherein t is an integer from 0 to 500; wherein v′ is 0, 1, 2, 3, or 4; wherein w is 0 or 1; wherein x is 0 or 1; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

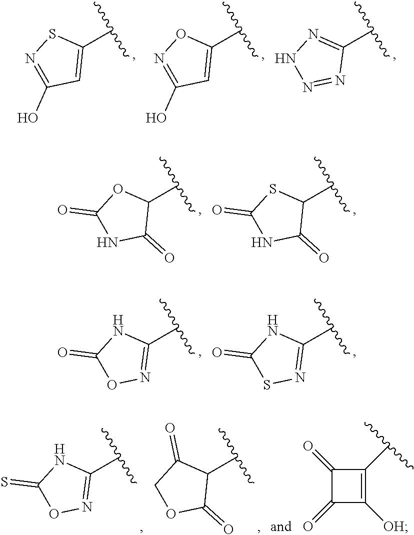

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, wherein at least one of the DNA barcodes is attached to at least one of the compounds.

Also, disclosed are deoxyribonucleic acid- (DNA-) encoded libraries comprising a plurality of complexes, wherein each complex comprises: (a) one or more DNA barcodes; and (b) one or more thioether or selenoether compounds having a structure represented by a formula:

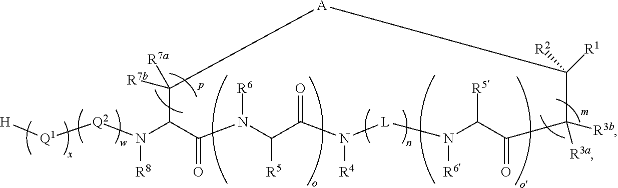

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein x is 0 or 1; wherein A is S or Se; wherein A is S or Se; wherein L, when present, is selected from C2-C4 alkyl, —(C1-C4 alkyl)(OCH₂CH₂)_{q}, and a structure selected from:

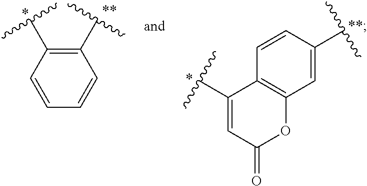

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

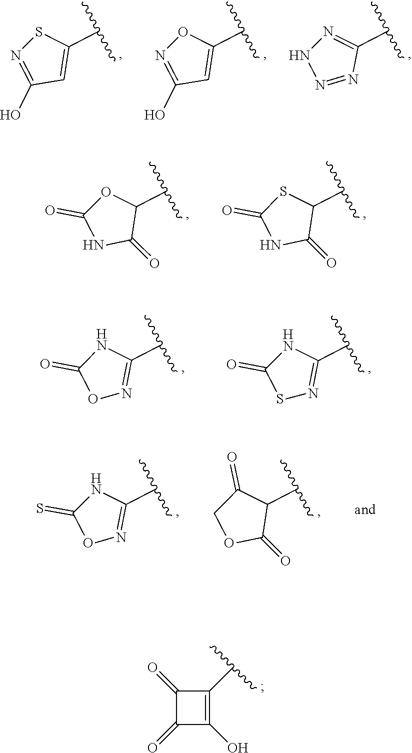

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, wherein at least one of the DNA barcodes is attached to at least one of the compounds.

Also disclosed are methods of screening using a DNA-encoded library, the method comprising: (a) incubating one or more compounds with a unique DNA barcode under conditions for attachment of each of the one or more compounds to the unique DNA barcode, whereby complexes comprising the unique DNA barcode and a compound are formed, wherein the one or more compounds has a structure represented by a formula:

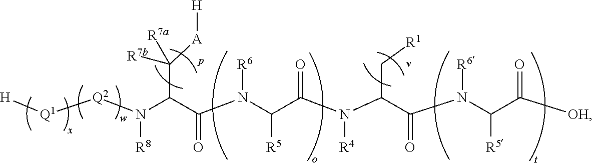

wherein o is 0, 1, 2, 3, 4, 5, 6, 7, 8, or 9; wherein p is 1 or 2; wherein t is an integer from 0 to 500; wherein v is 1, 2, 3, 4, or 5; wherein w is 0 or 1; wherein x is 0 or 1; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

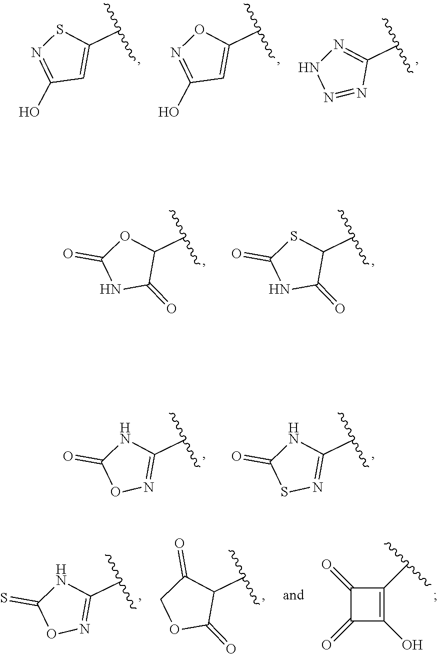

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, (b) contacting the complexes with PapB under conditions sufficient to allow the compound to form a thioether or selenoether crosslink, thereby forming a complex comprising a cross-linked compound; (c) incubating complexes comprising the cross-linked compound with one or more biological targets; (d) isolating the complexes comprising the cross-linked compound bound to the biological target; and (e) obtaining the sequence of the unique DNA barcode of each of the one or more complexes comprising the cross-linked compound bound to the biological target, wherein the sequence of the unique DNA barcode identifies the compound bound to the biological target.

Also disclosed are methods of screening using a DNA-encoded library, the method comprising: (a) incubating one or more compounds with a unique DNA barcode under conditions for attachment of each of the one or more compounds to the unique DNA barcode, whereby complexes comprising the unique DNA barcode and a compound are formed, wherein the one or more compounds has a structure represented by a formula:

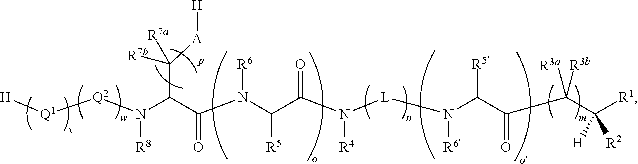

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein x is 0 or 1; wherein A is S or Se; wherein A is S or Se; wherein L, when present, is selected from C2-C4 alkyl, —(C1-C4 alkyl)(OCH₂CH₂)_{q}, and a structure selected from:

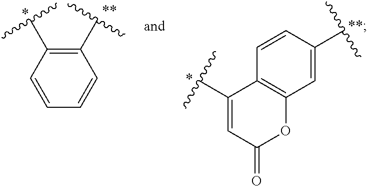

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

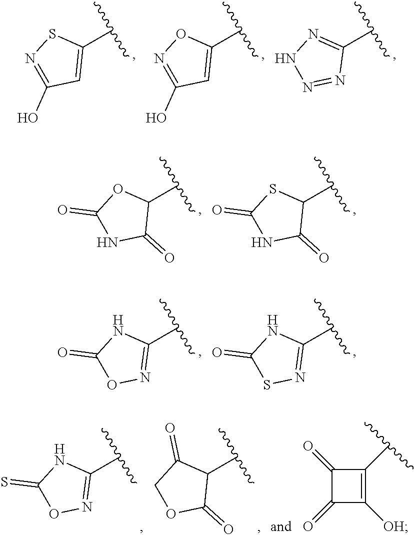

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, (b) contacting the complexes with PapB under conditions sufficient to allow the compound to form a thioether or selenoether crosslink, thereby forming a complex comprising a cross-linked compound; (c) incubating complexes comprising the cross-linked compound with one or more biological targets; (d) isolating the complexes comprising the cross-linked compound bound to the biological target; and (e) obtaining the sequence of the unique DNA barcode of each of the one or more complexes comprising the cross-linked compound bound to the biological target, wherein the sequence of the unique DNA barcode identifies the compound bound to the biological target.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the term “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease, disorder, or condition. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the condition being treated and the severity of the condition; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “sactipeptide” refers to a sulfur-to-alpha carbon thioether cross-linked peptide belonging to the ribosomally synthesized post-translationally modified peptide (RiPP) superfamily. As illustrated by the structure below, a sactipeptide contains an intramolecular thioether bond that crosslinks the sulfur atom of a cysteine residue to the α-carbon of an acceptor amino acid.

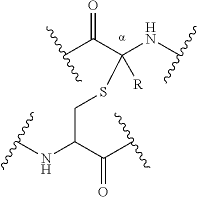

As used herein, the term “ranthipeptide” refers to a radical non-α thioether-containing peptide, which, similar to sactipeptides above, is also a member of the RiPP superfamily. For example, as illustrated below, a ranthipeptide can contain an intramolecular thioether bond that crosslinks the sulfur atom of a cysteine residue to any carbon other than the α-carbon of an acceptor amino acid.

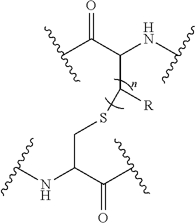

Exemplary ranthipeptide residues containing an β- or γ-carbon are shown below.

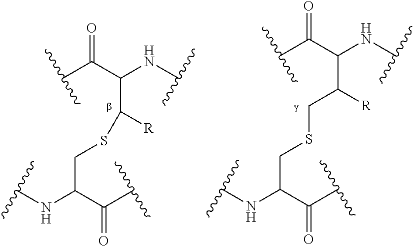

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula (CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide” as used herein can be used interchangeably and refer to F, Cl, Br, or I.

The terms “pseudohalide,” “pseudohalogen,” or “pseudohalo” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein, refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein, refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula -A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}—NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

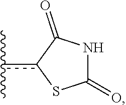

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di-, or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure. [00102]“Derivatives” of the compounds disclosed herein are pharmaceutically acceptable salts, prodrugs, deuterated forms, radio-actively labeled forms, isomers, solvates and combinations thereof. The “combinations” mentioned in this context refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

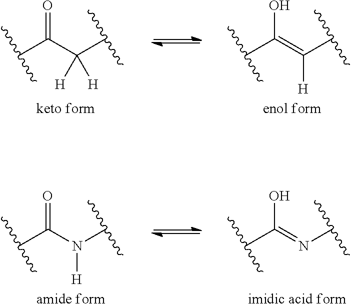 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

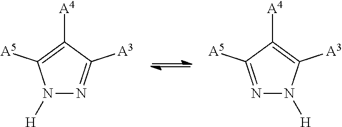

Unless stated to the contrary, the invention includes all such possible tautomers.

It is known that chemical substances form solids, which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In various aspects, a structure of a compound can be represented by a formula:

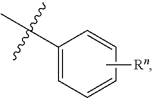

which is understood to be equivalent to a formula:

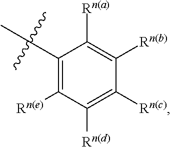

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a}) is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. DNA-ENCODED LIBRARIES (DELs)

In one aspect, disclosed are DNA-encoded libraries comprising a plurality of complexes, wherein each complex has a formula A-(B)ₓ—C, wherein A is a compound as disclosed herein; wherein B is an optional linker (i.e., x is 0 or 1); and C is a DNA barcode. Thus, in various aspects, the linker is absent. In various further aspects, the linker is present.

In one aspect, disclosed are DNA-encoded libraries comprising a plurality of complexes, wherein each complex comprises: (a) one or more DNA barcodes; and (b) one or more compounds having a structure represented by a formula:

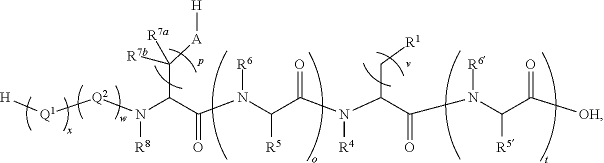

wherein o is 0, 1, 2, 3, 4, 5, 6, 7, 8, or 9; wherein p is 1 or 2; wherein t is an integer from 0 to 500; wherein v is 1, 2, 3, 4, or 5; wherein w is 0 or 1; wherein x is 0 or 1; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

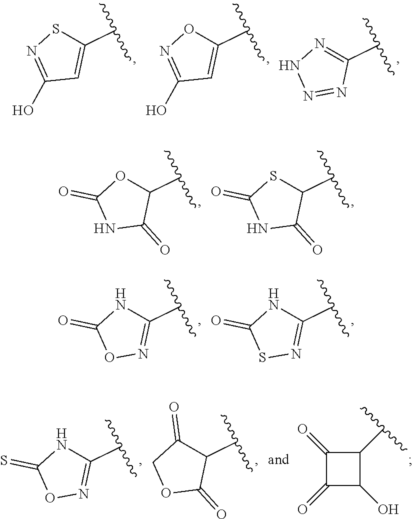

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, wherein at least one of the DNA barcodes is attached to at least one of the compounds.

In one aspect, disclosed are DNA-encoded libraries comprising a plurality of complexes, wherein each complex comprises: (a) one or more DNA barcodes; and (b) one or more compounds having a structure represented by a formula:

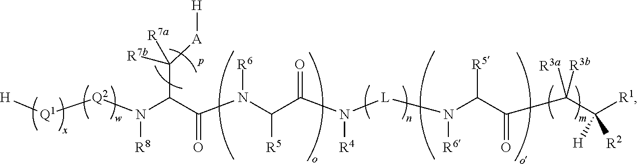

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein x is 0 or 1; wherein A is S or Se; wherein A is S or Se; wherein L, when present, is selected from C2-C4 alkyl, —(C1-C4 alkyl)(OCH₂CH₂)_{q}, and a structure selected from:

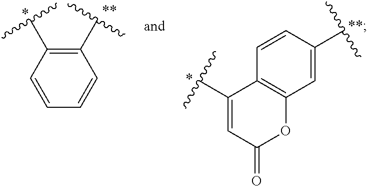

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

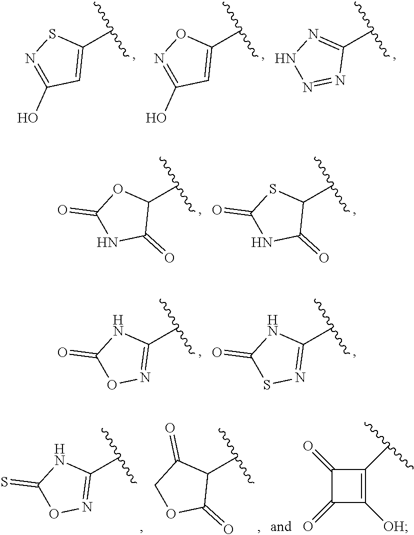

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, wherein at least one of the DNA barcodes is attached to at least one of the compounds.

In one aspect, disclosed are DNA-encoded libraries comprising a plurality of complexes, wherein each complex comprises: (a) one or more DNA barcodes; and (b) one or more thioether or selenoether compounds having a structure represented by a formula:

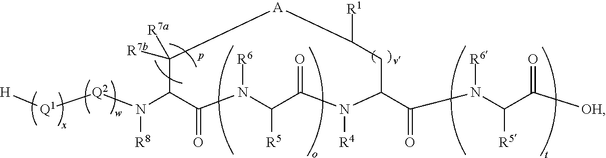

wherein o is 0, 1, 2, 3, 4, 5, 6, 7, 8, or 9; wherein p is 1 or 2; wherein t is an integer from 0 to 500; wherein v′ is 0, 1, 2, 3, or 4; wherein w is 0 or 1; wherein x is 0 or 1; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

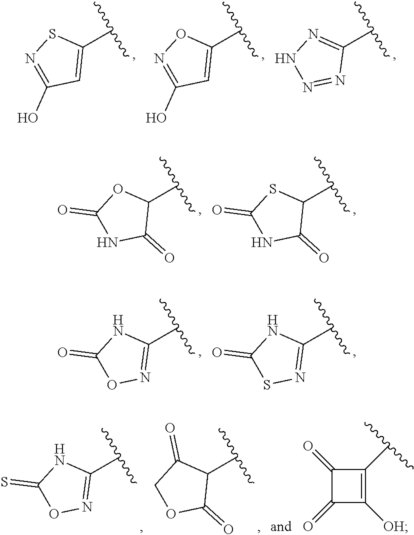

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C₄ alkyl; and wherein R⁸ is selected from hydrogen and methyl, wherein at least one of the DNA barcodes is attached to at least one of the compounds.

In one aspect, disclosed are DNA-encoded libraries comprising a plurality of complexes, wherein each complex comprises: (a) one or more DNA barcodes; and (b) one or more thioether or selenoether compounds having a structure represented by a formula:

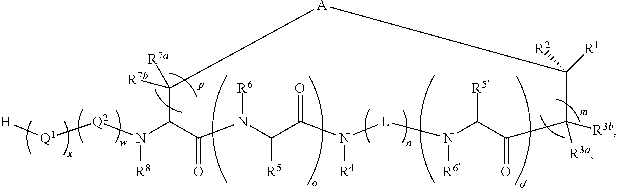

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein x is 0 or 1; wherein A is S or Se; wherein A is S or Se; wherein L, when present, is selected from C2-C4 alkyl, —(C1-C4 alkyl)(OCH₂CH₂)_{q}, and a structure selected from:

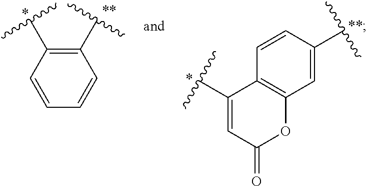

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

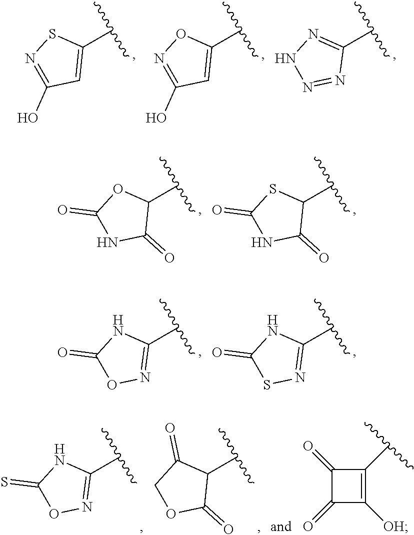

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, wherein at least one of the DNA barcodes is attached to at least one of the compounds.

Herein, the combination of DELs with enzymatic cross-linking is described, which significantly expands the accessible chemical space compared to the chemical space that is accessible through other display platforms such as phage display and mRNA display. Phage and mRNA display systems are inherently limited by the translation machinery, which requires that even when noncanonical amino acids are incorporated, the backbone remains fundamentally peptide-based and generally limited to structures that can be ribosomally incorporated. DELs bypass that restriction, enabling direct synthesis and screening of compounds containing multiple unnatural amino acids, entirely non-peptidic scaffolds, or even hybrid constructs that are enzymatically (hyper)modified before screening.

As detailed herein, a DNA-encoded library or DEL is an encoded combinatorial chemical library comprising a plurality of complexes that each define a different chemical structure (i.e., a different compound, thioether compound, or selenoether compound) and that each contain a unique identifier oligonucleotide whose nucleotide sequence defines the corresponding chemical structure. The complex is the basic unit in a library of this invention, and combines the elements of a peptide (e.g., a macrocyclic peptide) and a code for identifying the structure of the peptide. Thus, in various aspects, a complex can be represented by a formula A-B-C, where A is a disclosed compound (e.g., compound, thioether compound, or selenoether compound), B is an optional linker molecule operatively linked to A and C, and C is a DNA barcode comprising a sequence of nucleotides that identifies the structure of compound A.

The number of different species in a library represents the complexity of a library and is defined by the polymer length of the compound and by the size of the chemical unit alphabet that can be used to build the compound. The number of different species referred to by the phrase “plurality of species” in a library can be defined by the formula Vₐ, i.e., V to power of a (exponent a). V represents the alphabet size, i.e., the number of different chemical units X available for use in the compound. a is an exponent to V and represents the number of chemical units of X forming the compound, i.e., the length of the compound.

For example, for a complex where compound (A) is a peptide having a length of 6 amino acids, and where the amino acids utilized can be any of the 20 natural amino acids, the alphabet (V) is 20 and the polymer length (a) is 6, and the library size is 206 or 64 million. This exemplary library provides a repertoire of chemical diversity comprising 64 million different hexameric polypeptides operatively linked to corresponding unique DNA barcodes (identifier oligonucleotides).

Because the complexity of the library will determine the amount of a particular species of a complex relative to the other species in the library, there are theoretical limits to the maximum useful complexity in a library. Therefore, it is useful to consider how large (or complex) a library should be. This size limit is dictated by the level of sensitivity for detecting the presence of a compound DNA barcode after a screening procedure according to this invention. Detection sensitivity is dictated by the threshold of binding between an acceptor molecule to be assayed and a complex.

If, for example, the binding threshold is 10⁻⁶ M (micromolar), then there must be at least one nanomole of each complex species in a library of 1 milliliter (ml) volume. At this threshold, a library having a complexity of 104 could contain 10 micromoles of each complex species. Because of the reciprocal relationship between library complexity and binding threshold, more complex libraries are possible where the binding threshold is lower.

The relative amounts of the individual complex species within the library can vary from about 0.2 equivalents to about 10 equivalents, where an equivalent represents the average amount of a species within the library. In various aspects, each complex species is present in the library in approximately equimolar amounts.

In various aspects, a library contains the complete repertoire of chemical diversity possible based on the mathematical combinations for a given library where there is a fixed alphabet and a preselected number of chemical units in all species of the library. Thus, a complete repertoire is one that provides a source of all the possible chemical diversity that can be found in a library of this invention having a fixed alphabet and chemical length.

In various aspects, the library is comprised of complexes where each species of complex contains the same nucleotide sequence for either the P1 or P2 PCR primer binding sites. A library with this design is particularly preferred because, when practicing the methods of this invention, a single PCR primer pair can be used to amplify any species of DNA barcode (coding sequence) present in the library.

As would be understood by one of ordinary skill in the art, the DNA barcode can be attached to the N-terminus or the C-terminus of the compound or, alternatively, can be attached to a specific side chain (e.g., cysteine, lysine, tyrosine) of the compound. Thus, in various aspects, the DNA barcode is attached to the N-terminus of the compound. In various further aspects, the DNA barcode is attached to the C-terminus of the compound. In various further aspects, the DNA barcode is attached to a side chain of the compound.

Methods of attaching a DNA barcode to a compound are well known in the art. See, e.g., Liszczak and Muir (2020) Angew. Chem. Int. Ed. Engl. 58(13): 4144-4162. Thus, in various aspects, a DNA barcode can be attached to the compound via chemical conjugation, where the barcode (typically, a modified oligonucleotide) is linked to a specific amino acid residue or functional group on the compound. Alternative techniques for attaching the compound include, but are not limited to, Edman degradation, ligase-mediated ligation, and transposase-mediated transposition. In this way, identification and tracking of the compound in complex moistures and/or during chemical reactions is facilitated.

In various aspects, each DNA barcode is unique to a single compound.

In various aspects, each DNA barcode is 5 to 100 nucleotides in length. In a further aspect, each DNA barcode is 10 to 95 nucleotides in length. In a still further aspect, each DNA barcode is 15 to 90 nucleotides in length. In yet a further aspect, each DNA barcode is 15 to 85 nucleotides in length. In an even further aspect, each DNA barcode is 20 to 80 nucleotides in length. In an even still further aspect, each DNA barcode is 25 to 75 nucleotides in length. In yet an even further aspect, each DNA barcode is 30 to 70 nucleotides in length. In a further aspect, each DNA barcode is 35 to 65 nucleotides in length. In a still further aspect, each DNA barcode is 40 to 60 nucleotides in length. In yet a further aspect, each DNA barcode is 45 to 55 nucleotides in length.

In various aspects, at least one of the DNA barcodes attached to at least one of the compounds comprises at least one of the DNA barcodes conjugated or linked to at least one of the compounds.

In various aspects, the complex further comprises a linker between at least one of the DNA barcodes and at least one of the compounds.

### 1. DNA Barcodes

In various aspects, A DNA barcode comprises an oligonucleotide having a sequence that can be used to identify the compound that the DNA barcode is linked to and can comprise primer binding sites that allow for amplification of the DNA barcode.

The length of a DNA barcode can vary depending on the complexity of the library, the number of different compounds to be uniquely identified, and other considerations relating to requirements for uniqueness of oligonucleotides such as hybridization and polymerase chain reaction fidelity. A typical length can be from about 2 to about 10 nucleotides, although nothing is to preclude a DNA barcode from being longer. In various aspects, the DNA barcode can be at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In various aspects, the DNA barcode can be at least 5, 10, 15, 20, 25, 30, 35, 40, 45, or 50 nucleotides in length.

In various aspects, adenosine (A), guanosine (G), thymidine (T) and cytosine (C) represent the typical choices of nucleotides for inclusion in a DNA barcode. A, G, T and C can form a representative “alphabet” used to “spell” out a DNA barcode's sequence. Other nucleotides or nucleotide analogs can be utilized in addition to or in place of the above four nucleotides, so long as they have the ability to form Watson-Crick pairs and be replicated by DNA polymerases in a PCR reaction.

In various aspects, for the design of the sequence in the DNA barcode, it is essential to choose a coding representation such that no significant part of the oligonucleotide sequence can occur in another unrelated combination by chance or otherwise during the manipulations of a complex in the library.

Other sources of misreading of a unit identifier oligonucleotide can arise. For example, mismatch in DNA hybridization, transcription errors during a primer extension reaction to amplify or sequence the identifier oligonucleotide, and the like errors can occur during a manipulation of a bifunctional molecule.

In various aspects, a variety of means to reduce the possibility of error in reading the DNA barcode can be used, such as to use longer nucleotide lengths to reduce the similarity between DNA barcode sequences. Typical lengths can depend on the number of compounds in the library.

In various aspects, a DNA barcode has primer binding sites that allow for amplification of the DNA barcode which contains a sequence that is used to identify the compound the DNA barcode is linked to. The requirements for PCR primer binding sites are generally well known in the art, but are designed to allow a PCR amplification product (a PCR-amplified duplex DNA fragment) to be formed that contains the sequence used to identify the compound the DNA barcode is linked to. In various aspects, there are two primer binding sites that flank sequence that is used to identify the compound the DNA barcode is linked to. In various aspects, the primer binding sites are designed to contain a means for removing the primer binding sites from the sequence used to identify the compound the DNA barcode is linked to. In various aspects, means for removing the primer binding sites can be restriction enzyme sites. Restriction endonucleases are well known in the art and are enzymes that recognize specific lengths of duplex DNA and cleave the DNA in a sequence-specific manner.

### 2. Linkers

In one aspect, the disclosed DNA-encoded libraries comprise a plurality of complexes, wherein each complex optionally comprises one or more linkers.

A linker can be any molecule that performs the function of operatively linking the compound to the DNA barcode. In various aspects, the phrase “operatively linking” can be a means for combining or connecting two or more elements in a manner that allows them to work together. In various aspects, operatively linking can be used interchangeably with “functionally connecting”.

In various aspects, a linker can comprise a means for attaching to a solid support. In various aspects, the means for attaching a linker to a solid support is reversible. For example, in various aspects, the linker, and thus the compound and DNA barcode to which the linker is conjugated, can be separated from the solid support.

A linker can vary in structure and length, and provide at least two features: (1) operative linkage to the compound, and (2) operative linkage to the DNA barcode. As the nature of chemical linkages is diverse, any of a variety of chemistries may be utilized to effect the indicated operative linkages to the compound and the DNA barcode, as the nature of the linkage is not considered an essential feature of this invention. The size of the linker in terms of the length between the compound and the DNA barcode can vary widely, but for the purposes of the invention, need not exceed a length sufficient to provide the linkage functions indicated. Thus, a chain length of from at least one to about 50 nucleotides is preferred.

### 3. Compounds

In one aspect, the disclosed DNA-encoded libraries comprise a plurality of complexes, wherein each complex comprises one or more compounds having a structure represented by a formula:

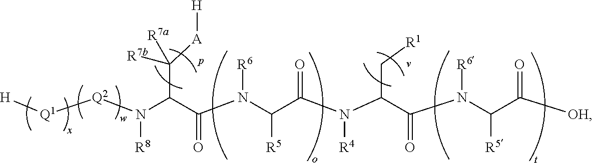

wherein o is 0, 1, 2, 3, 4, 5, 6, 7, 8, or 9; wherein p is 1 or 2; wherein t is an integer from 0 to 500; wherein v is 1, 2, 3, 4, or 5; wherein w is 0 or 1; wherein x is 0 or 1; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

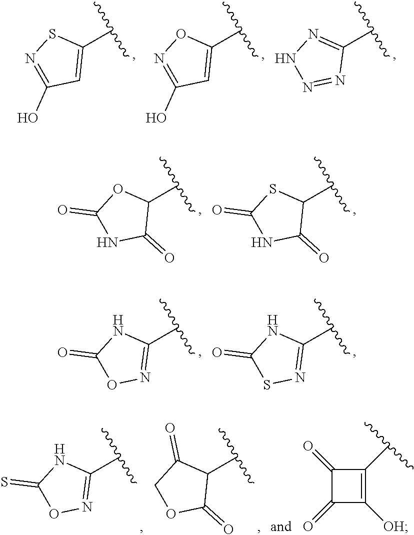

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, wherein at least one of the DNA barcodes is attached to at least one of the compounds.

In one aspect, disclosed are compounds having a structure represented by a formula:

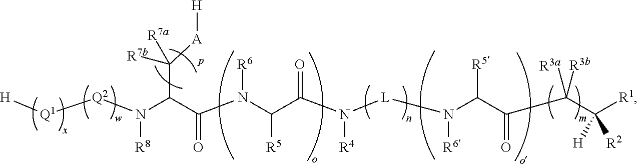

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein x is 0 or 1; wherein A is S or Se; wherein A is S or Se; wherein L, when present, is selected from C2-C4 alkyl, —(C1-C4 alkyl)(OCH₂CH₂)_{q}, and a structure selected from:

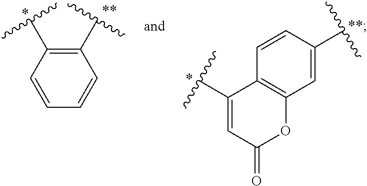

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

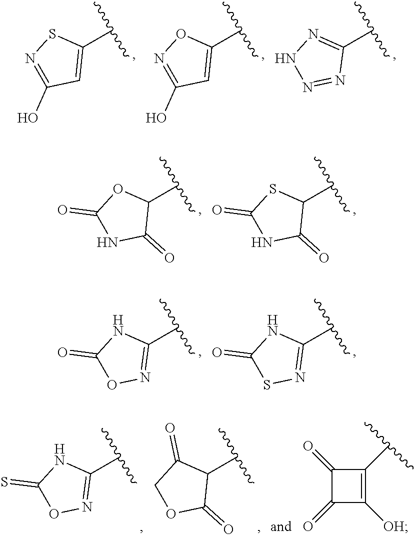

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, wherein at least one of the DNA barcodes is attached to at least one of the compounds.

In various aspects, the compound has a structure represented by a formula:

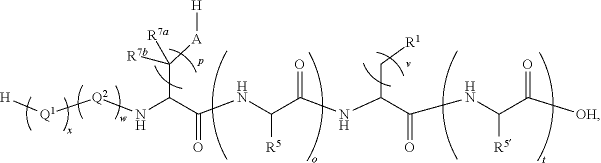

In various aspects, the compound has a structure represented by a formula:

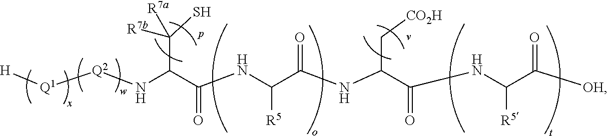

In various aspects, the compound has a structure represented by a formula:

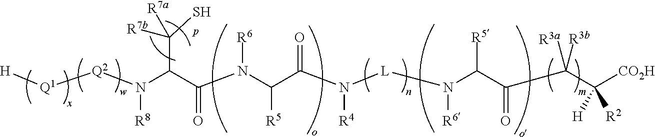

In various aspects, the compound has a structure represented by a formula:

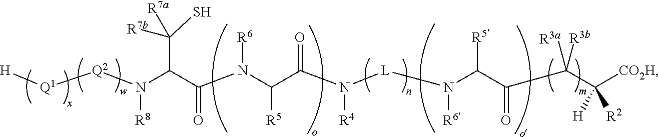

In various aspects, the compound has a structure represented by a formula:

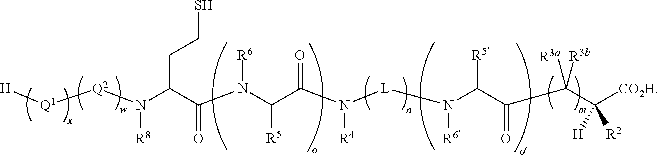

In various aspects, the compound has a structure represented by a formula:

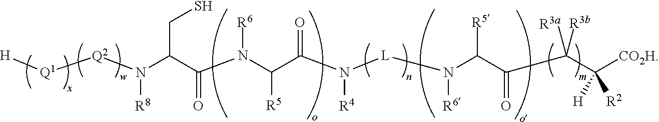

In various aspects, the compound has a structure represented by a formula:

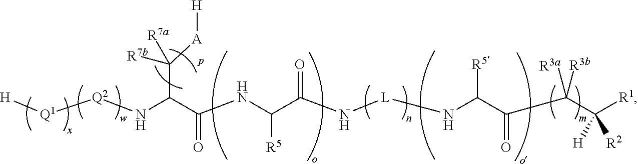

In various aspects, the compound has a structure represented by a formula:

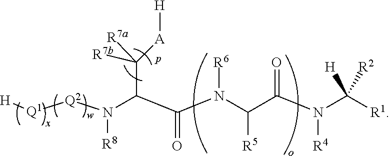

In various aspects, the compound has a structure represented by a formula:

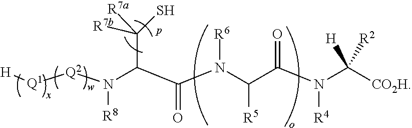

In various aspects, the compound has a structure represented by a formula:

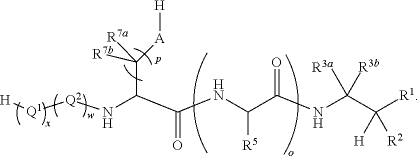

In a further aspect, o is 1, 2, 3, 4, 5, 6, 7, 8, or 9.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

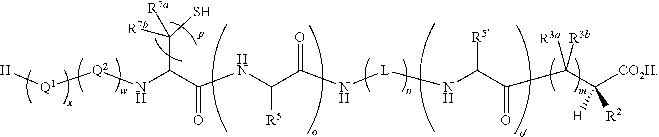

In various aspects, the compound has a structure represented by a formula:

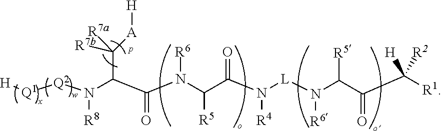

In various aspects, the compound has a structure represented by a formula:

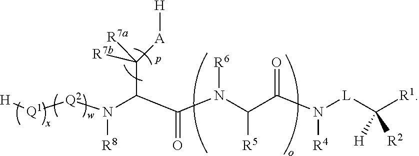

In various aspects, the compound has a structure represented by a formula:

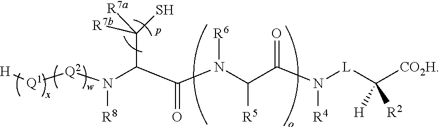

In a further aspect, o is 1, 2, 3, 4, 5, 6, 7, 8, or 9.

In various aspects, the compound has a structure represented by a formula:

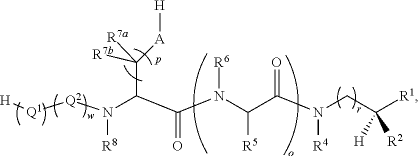

wherein r is 2, 3, or 4.

In various aspects, the compound has a structure represented by a formula:

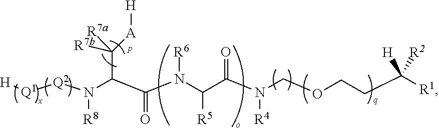

wherein s is 1 or 2.

In various aspects, the compound has a structure represented by a formula:

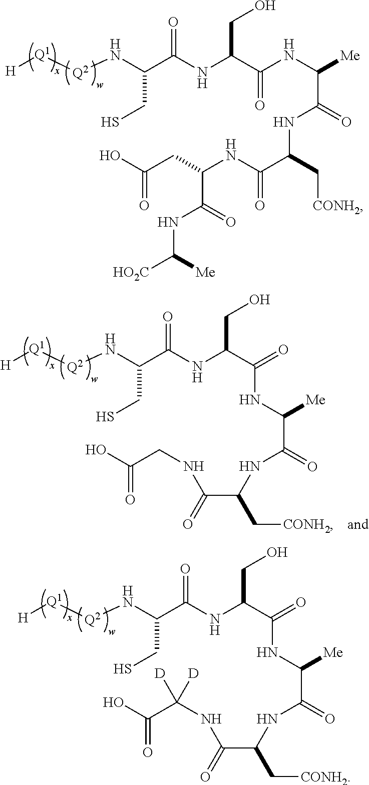

In various aspects, m is 0, 1, 2, 3, or 4. In a further aspect, m is 0, 1, 2, or 3. In a still further aspect, m is 0, 1, or 2. In yet a further aspect, m is 0 or 1. In an even further aspect, m is 0. In an even still further aspect, m is 1. In yet an even further aspect, m is 2. In a further aspect, m is 3. In a still further aspect, m is 4.

In various aspects, n is 0 or 1. In a further aspect, n is 0. In yet a further aspect, n is 1.

In various aspects, o is 0, 1, 2, 3, 4, 5, 6, 7, 8, or 9. In a further aspect, o is 0, 1, 2, 3, 4, 5, 6, 7, or 8. In a still further aspect, o is 0, 1, 2, 3, 4, 5, 6, or 7. In an even yet further aspect, o is 0, 1, 2, 3, 4, 5, or 6. In a further aspect, o is 0, 1, 2, 3, 4, or 5. In a still further aspect, o is 0, 1, 2, 3, or 4. In a yet further aspect, o is 0, 1, 2, or 3. In an even further aspect, o is 0, 1, or 2. In a still further aspect, o is 0 or 1.

In various aspects, o is an integer from 0 to 5. In a further aspects, o is 5. In a still further aspect, o is 4. In yet a further aspect, o is 3. In an even further aspect, o is 2. In yet an even further aspect, o is 1

In various aspects, each of o and o′ is independently 0, 1, 2, 3, 4, 5, 6, 7, 8, or 9. In a further aspect, each of o and o′ is independently 0, 1, 2, 3, 4, 5, 6, 7, or 8. In a still further aspect, each of o and o′ is independently 0, 1, 2, 3, 4, 5, 6, or 7. In yet a further aspect, each of o and o′ is independently 0, 1, 2, 3, 4, 5, or 6. In an even further aspect, each of o and o′ is independently 0, 1, 2, 3, 4, or 5. In a still further aspect, each of o and o′ is independently 0, 1, 2, 3, or 4. In yet a further aspect, each of o and o′ is independently 0, 1, 2, or 3. In an even further aspect, each of o and o′ is independently 0, 1, or 2. In a still further aspect, each of o and o′ is independently 0 or 1.

In various aspects, p is 1 or 2. In a further aspect, p is 1. In yet a further aspect, p is 2.

In various aspects, q is 1, 2, 3, or 4. In a further aspect, q is 1, 2, or 3. In a still further aspect, q is 1 or 2. In yet a further aspect, q is 1.

In various aspects, r is an integer from 7 to 50. In a further aspect, r is an integer from 15 to 45. In a still further aspect, r is an integer from 20 to 40. In yet a further aspect, r is an integer from 25 to 35. In an even further aspect, r is an integer from 26 to 34. In a still further aspect, r is an integer from 27 to 33. In yet a further aspect, r is an integer from 28 to 32. In an even further aspect, r is an integer from 29 to 31. In an even still further aspect, r is 31.

In various aspects, s is 0 or 1. In a further aspect, s is 0. In a still further aspect, s is 1.

In various aspects, u is 0 or 1. In a further aspect, u is 0. In a still further aspect, u is 1.

In various aspects, t is an integer from 0 to 500. In a further aspect, t is an integer from 0 to 250. In a still further aspect, t is an integer from 0 to 100. In yet a further aspect, t is an integer from 0 to 50. In an even further aspect, t is an integer from 0 to 25. In a still further aspect, t is an integer from 0 to 10. In yet a further aspect, t is 0, 1, 2, 3, 4, or 5. In an even further aspect, t is 0, 1, 2, or 3. In a still further aspect, t is 0 or 1. In yet a further aspect, t is 0.

In various aspects, v is 1, 2, 3, 4, or 5. In a still further aspect, v is 1, 2, 3, or 4. In yet a further aspect, v is 1, 2, or 3. In an even further aspect, v is 1 or 2. In a still further aspect, v is 1 or 2. In yet a further aspect, v is 1. In an even further aspect, v is 2.

In various aspects, w is 0 or 1. In a further aspect, w is 0. In a still further aspect, w is 1.

In various aspects, w′ is 1, 2, 3, 4, 5, or 6. In a further aspect, w′ is 1, 2, 3, 4, or 5. In a still further aspect, w′ is 1, 2, 3, or 4. In yet a further aspect, w′ is 1, 2, or 3. In an even further aspect, w′ is 1, or 2. In a still further aspect, w′ is 1.

In various aspects, x is 0 or 1. In a further aspect, x is 0. In a still further aspect, x is 1.

a. A Groups

In one aspects, A is S or Se. In a further aspect, A is S. In a still further aspect, A is Se.

b. L Groups

In one aspect, L is selected from C2-C4 alkyl, —(C1-C4 alkyl)(OCH₂CH₂)_{q}, and a structure selected from:

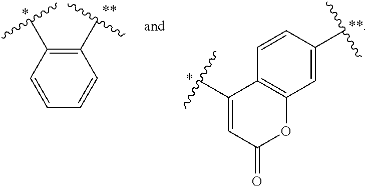 [SMILES: *c1ccccc1* ; *c1ccc2c(*)cc(=O)oc2c1]

In various aspects, L is selected from C2-C4 alkyl and —(C1-C4 alkyl)(OCH₂CH₂)_{q}. In a further aspect, L is selected from methyl, ethyl, n-propyl, isopropyl, —CH₂(OCH₂CH₂)_{q}, —CH₂CH₂(OCH₂CH₂)_{q}, —CH₂CH₂CH₂(OCH₂CH₂)_{q}, and —CH(CH₃)(CH₂(OCH₂CH₂)_{q}). In a still further aspect, L is selected from methyl, ethyl, —CH₂(OCH₂CH₂)_{q}, and —CH₂CH₂(OCH₂CH₂)_{q}. In yet a further aspect, L is selected from methyl and —CH₂(OCH₂CH₂)_{q}.

In various aspects, L is a structure selected from:

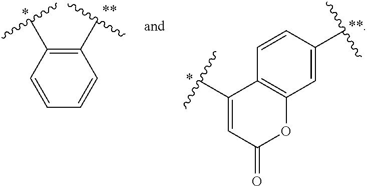 [SMILES: *c1ccccc1* ; *c1ccc2c(*)cc(=O)oc2c1]

c. Q¹ Groups

In one aspect, Q¹ is a leader sequence. In a further aspect, the leader sequence is LKQINVIAGVKEPIRAYG (SEQ ID NO: 2) or LKQINVIAGVKPIRAYG (SEQ ID NO: 3). In a yet further aspect, the leader sequence is LKQINVIAGVKEPIRAYG (SEQ ID NO: 2).

d. Q² Groups

In one aspect, Q² is a chemically cleavable moiety. Exemplary chemical cleavable moieties include, but are not limited to, —CO₂—(C4-C8 alkylene)-OC(O)—. In a further aspect, the chemically cleavable moiety is —CO₂CH₂CH═CHCH₂OC(O)—.

In various aspects, the cleavable moiety is an enzymatically cleavable moiety such as, for example, a protease recognition sequence. In a further aspect, the protease recognition sequence is TEV recognition sequence. In a further aspect, the TEV protease recognition sequence is EXLYZQ (SEQ ID NO: 4), in which X is any amino acid and Z is any amino acid that contains a hydrophobic residue. In yet a further aspect, the TEV protease recognition sequence is ENLYFQ (SEQ ID NO: 1).

e. R¹ Groups

In one aspect, R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

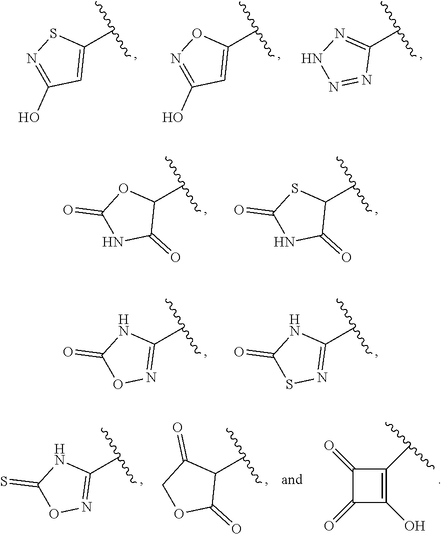

In various aspects, R¹ is selected from —CO₂H, —C(O)NHOH, —SO₂NH₂, —SO₂NHC(O)CH₃, —SO₃H, —NHC(O)NHSO₂CH₃, and —P(O)(OH)₂. In a further aspect, R¹ is selected from —CO₂H, —C(O)NHOH, —SO₂NH₂, and —SO₂NHC(O)CH₃. In a still further aspect, R¹ is selected from —CO₂H and —C(O)NHOH. In yet a further aspect, R¹ is —CO₂H.

In various aspects, R¹ is a structure selected from:

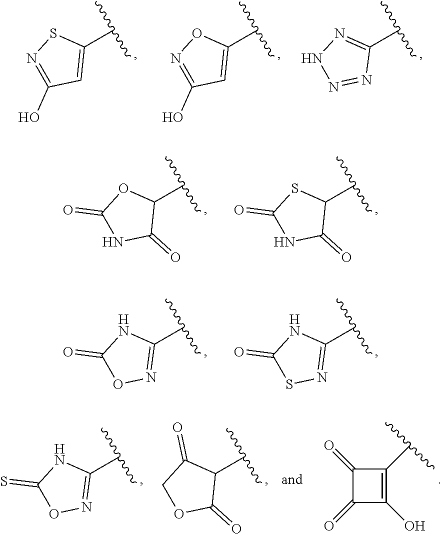

In various aspects, R¹ is —CO₂H or a structure:

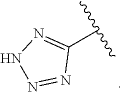 [SMILES: *c1nn[nH]n1]

In various aspects, R¹ is —CO₂H.

In various aspects, R¹ is a structure:

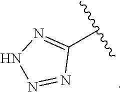 [SMILES: *c1nn[nH]n1]

f. R² Groups

In one aspect, R² is a residue of a side chain of amino acid, provided that the amino acid is not isoleucine or threonine. Exemplary residues of a side chain of an amino acid include, but are not limited to, natural amino acid residues, unnatural amino acid residues, and D-amino acid residues. Thus, in various aspects, R² is a D-amino acid residue. In various further aspects, R² is a β-amino acid residue. In various further aspects, R² is an N-methylated amino acid residue.

Natural amino acid residues may include glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine.

Unnatural amino acid residues may include, but are not limited to, p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenylalanine. Also, a p-propargyloxyphenylalanine, a 3,4-dihydroxy-L-phenyalanine (DHP), a 3,4,6-trihydroxy-L-phenylalanine, a 3,4,5-trihydroxy-L-phenylalanine, 4-nitro-phenylalanine, a p-acetyl-L-phenylalanine, O-methyl-L-tyrosine, an L-3-(2-naphthyl)alanine, a 3-methyl-phenylalanine, an O-4-allyl-L-tyrosine, a 4-propyl-L-tyrosine, a 3-nitro-tyrosine, a 3-thiol-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

g. R^{3A} and R^{3B} Groups

In one aspect, each of R^{3a} and R^{3b}, when present, is independently selected from C2-C5 alkynyl, C1-C5 azido, and a residue of a side chain of an amino acid.

In various aspects, each of R^{3a} and R^{3b}, when present, is independently selected ethynyl, proprynyl, butynyl, methyl azide, ethyl azide, n-propyl azide, isopropylazide, n-butyl azide, isobutyl azide, and tert-butyl azide. In a further aspect, each of R^{3a} and R^{3b}, when present, is independently selected ethynyl, proprynyl, methyl azide, ethyl azide, n-propyl azide, and isopropylazide. In a still further aspect, each of R^{3a} and R^{3b}, when present, is independently selected ethynyl, methyl azide, and ethyl azide.

In various aspects, each of R^{3a} and R^{3b}, when present, is independently a residue of a side chain of an amino acid. Exemplary residues of a side chain of an amino acid include, but are not limited to, natural amino acid residues, unnatural amino acid residues, and D-amino acid residues. Thus, in various aspects, at least one of R^{3a} and R^{3b} is a D-amino acid residue. In various further aspects, at least one of R^{3a} and R^{3b} is a p-amino acid residue. In various further aspects, at least one of R^{3a} and R^{3b} is an N-methylated amino acid residue.

Natural amino acid residues may include glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine.

Unnatural amino acid residues may include, but are not limited to, p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenylalanine. Also, a p-propargyloxyphenylalanine, a 3,4-dihydroxy-L-phenyalanine (DHP), a 3,4,6-trihydroxy-L-phenylalanine, a 3,4,5-trihydroxy-L-phenylalanine, 4-nitro-phenylalanine, a p-acetyl-L-phenylalanine, O-methyl-L-tyrosine, an L-3-(2-naphthyl)alanine, a 3-methyl-phenylalanine, an O-4-allyl-L-tyrosine, a 4-propyl-L-tyrosine, a 3-nitro-tyrosine, a 3-thiol-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

h. R⁴ Groups

In one aspect, R⁴ is selected from hydrogen and methyl. In a further aspect, R⁴ is hydrogen. In a still further aspect, R⁴ is methyl.

i. R⁵ and R^{5′} Groups

In one aspect, each occurrence of R⁵ and R^{5′}, when present, is independently a residue of a side chain of amino acid.

Exemplary residues of a side chain of an amino acid include, but are not limited to, natural amino acid residues, unnatural amino acid residues, and D-amino acid residues.

Natural amino acid residues may include glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine.

Unnatural amino acid residues may include, but are not limited to, p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenylalanine. Also, a p-propargyloxyphenylalanine, a 3,4-dihydroxy-L-phenyalanine (DHP), a 3,4,6-trihydroxy-L-phenylalanine, a 3,4,5-trihydroxy-L-phenylalanine, 4-nitro-phenylalanine, a p-acetyl-L-phenylalanine, O-methyl-L-tyrosine, an L-3-(2-naphthyl)alanine, a 3-methyl-phenylalanine, an O-4-allyl-L-tyrosine, a 4-propyl-L-tyrosine, a 3-nitro-tyrosine, a 3-thiol-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

j. R⁶ and R^{6′} Groups

In one aspect, each occurrence of R⁶ and R^{6′}, when present, is independently selected from hydrogen and methyl, or R⁶ and R^{6′}, when present, is covalently bonded to R⁵ and R^{5′}, respectively, when present, and, together with the intermediate atoms, comprise an unsubstituted 5-membered heterocycle.

In various aspects, each occurrence of R⁶, R^{6′}, and R^{6″}, when present, is independently selected from hydrogen and methyl. In a further aspect, each occurrence of R⁶ and R^{6′}, when present, is hydrogen. In a still further aspect, each occurrence of R⁶ and R^{6′}, when present, is methyl.

In various aspects, R⁶ and R^{6′}, when present, is covalently bonded to R⁵ and R^{5′}, respectively, when present, and, together with the intermediate atoms, comprise an unsubstituted 5-membered heterocycle.

k. R^{7A} and R^{7B} Groups

In one aspect, each of R^{7a} and R^{7b}, when present, is independently selected from hydrogen and C1-C4 alkyl. In a further aspect, each of R^{7a} and R^{7b}, when present, is independently selected from hydrogen, methyl, ethyl, propyl, and isopropyl. In a still further aspect, each of R^{7a} and R^{7b}, when present, is independently selected from hydrogen, methyl, and ethyl. In a yet further aspect, each of R^{7a} and R^{7b}, when present, is independently selected from hydrogen and methyl.

In various aspects, each of R^{7a} and R^{7b}, when present, is independently C1-C4 alkyl. In a further aspect, each of R^{7a} and R^{7b}, when present, is independently selected from methyl, ethyl, propyl, and isopropyl. In a still further aspect, each of R^{7a} and R^{7b}, when present, is independently selected from methyl and ethyl. In a yet further aspect, each of R^{7a} and R^{7b}, when present, is methyl.

In various aspects, each of R^{7a} and R^{7b}, when present, is hydrogen.

### 1. R⁸ Groups

In one aspect, R⁸ is selected from hydrogen and methyl. In a further aspect, R¹ is methyl. In a further aspect, R⁸ is hydrogen.

### 4. Thioether Compounds

In one aspect, the disclosed DNA-encoded libraries comprise a plurality of complexes, wherein each complex comprises one or more thioether or selenoether compounds having a structure represented by a formula:

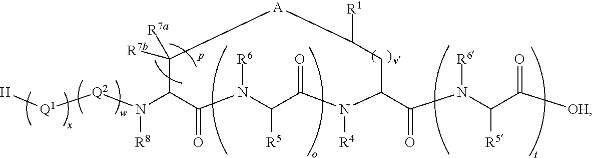

wherein o is 0, 1, 2, 3, 4, 5, 6, 7, 8, or 9; wherein p is 1 or 2; wherein t is an integer from 0 to 500; wherein v′ is 0, 1, 2, 3, or 4; wherein w is 0 or 1; wherein x is 0 or 1; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

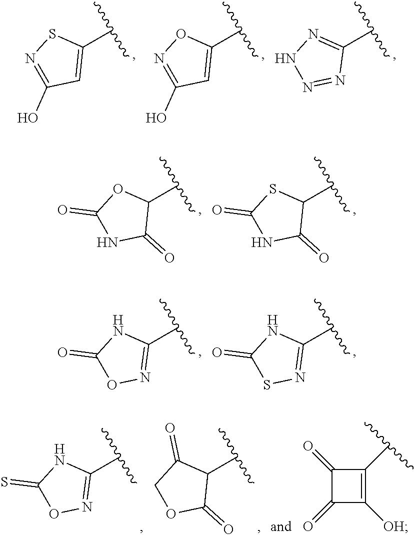

wherein R⁴ is selected from hydrogen and methyl; wherein each occurrence of R⁵ and R^{5′} when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, wherein at least one of the DNA barcodes is attached to at least one of the compounds.

In one aspect, disclosed are thioether or selenoether compounds having a structure represented by a formula:

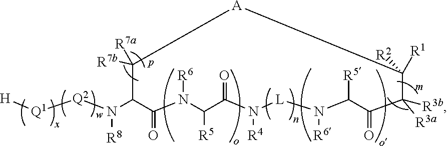

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein x is 0 or 1; wherein A is S or Se; wherein A is S or Se; wherein L, when present, is selected from C2-C4 alkyl, —(C1-C4 alkyl)(OCH₂CH₂)_{q}, and a structure selected from:

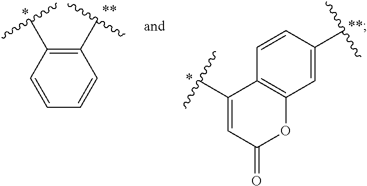

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

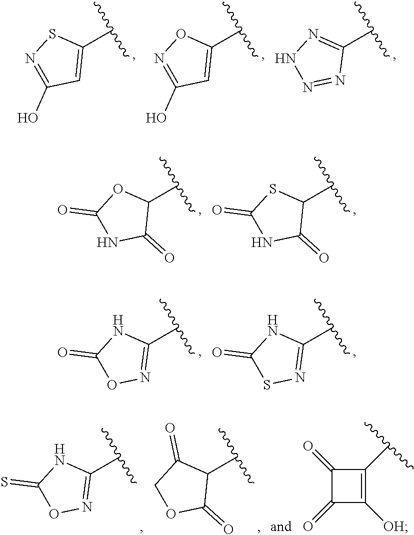

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, wherein at least one of the DNA barcodes is attached to at least one of the compounds.

In various aspects, the method produces a thioether or selenoether compound having a structure represented by a formula:

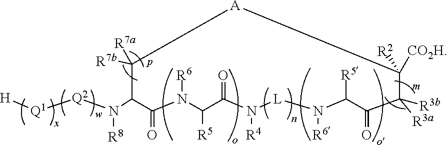

In various aspects, the thioether compound has a structure represented by a formula:

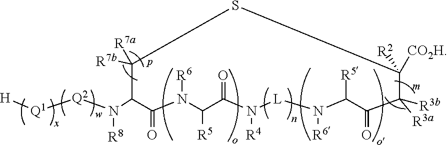

In various aspects, the thioether or selenoether compound has a structure represented by a formula:

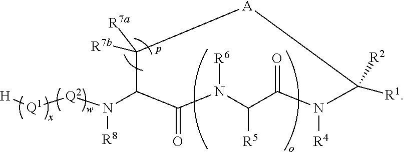

In various aspects, the thioether compound has a structure represented by a formula:

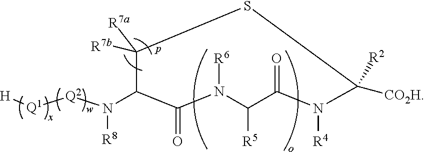

In various aspects, the thioether or selenoether compound has a structure represented by a formula:

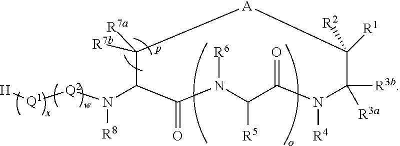

In various aspects, the thioether or selenoether compound has a structure represented by a formula:

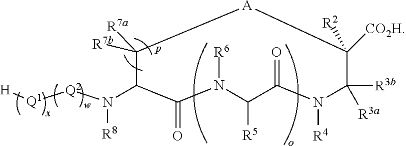

In various aspects, the thioether or selenoether compound has a structure represented by a formula:

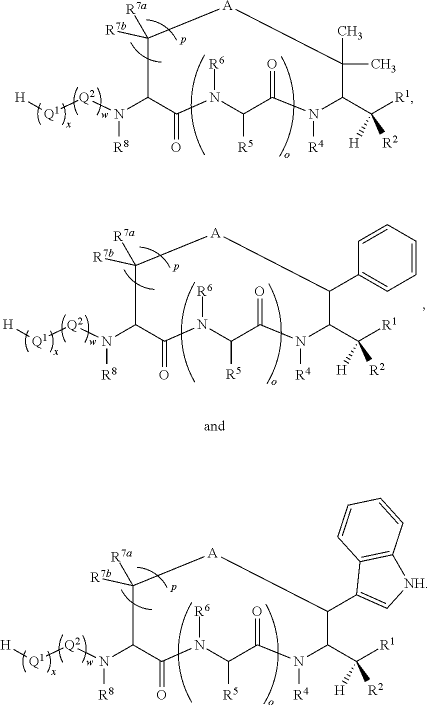

In various aspects, the thioether compound has a structure represented by a formula:

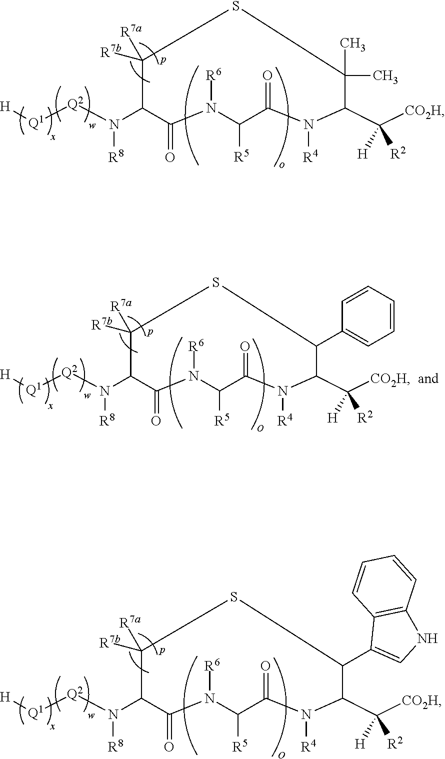

In various aspects, the thioether or selenoether compound has a structure represented by a formula:

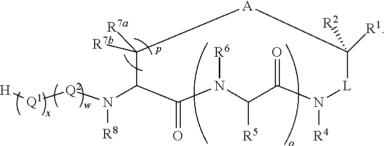

In various aspects, the thioether compound has a structure represented by a formula:

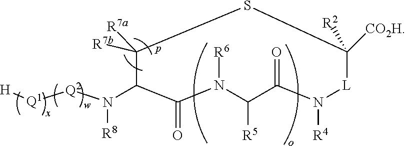

In various aspects, the thioether compound has a structure represented by a formula selected from:

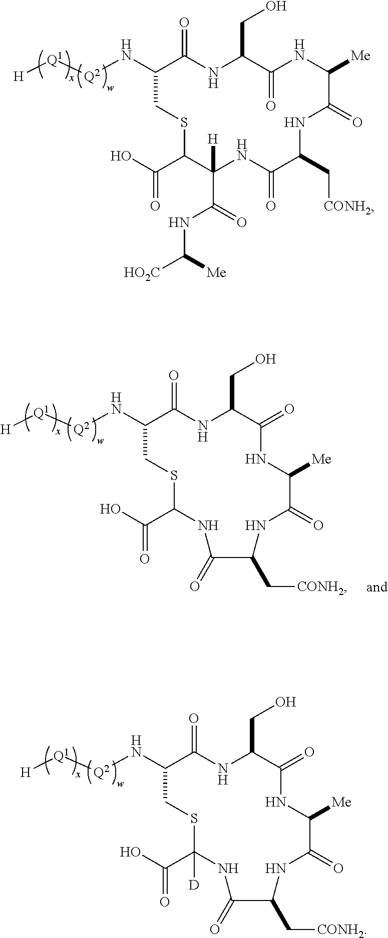

In various aspects, the thioether compound has a structure represented by a formula selected from:

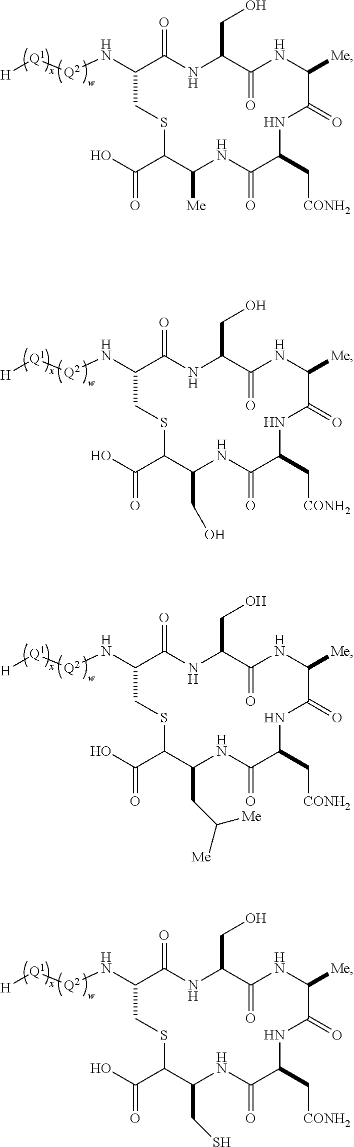

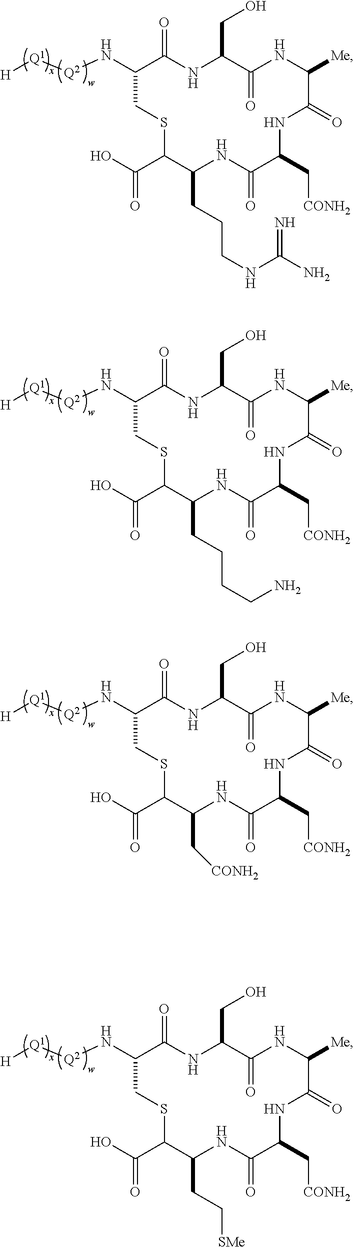

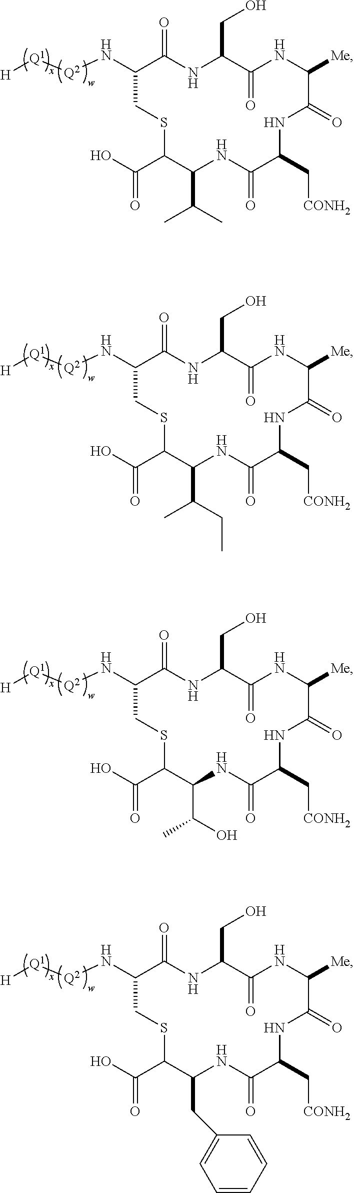

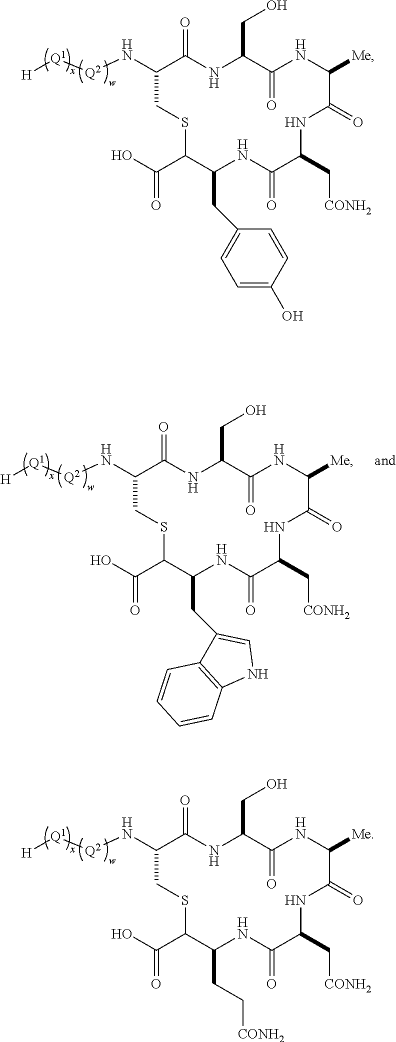

In various aspects, the thioether compound has a structure represented by a formula selected from:

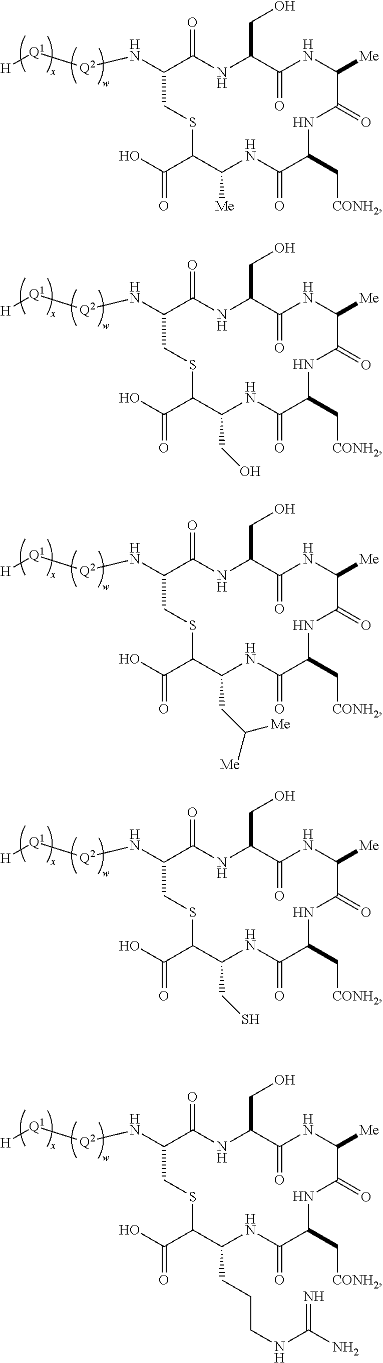

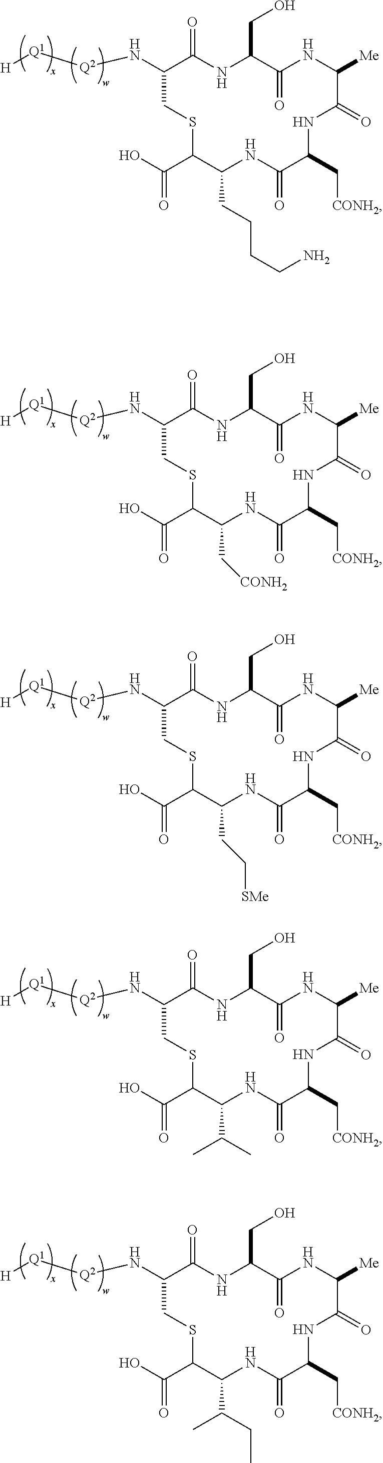

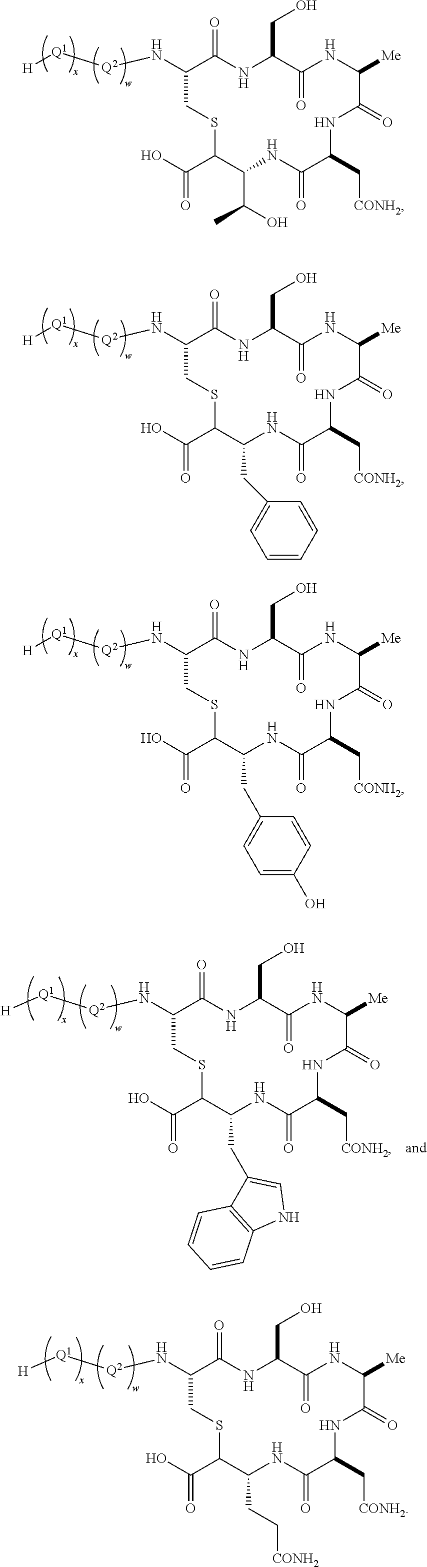

In various aspects, the thioether compound has a structure represented by a formula selected from:

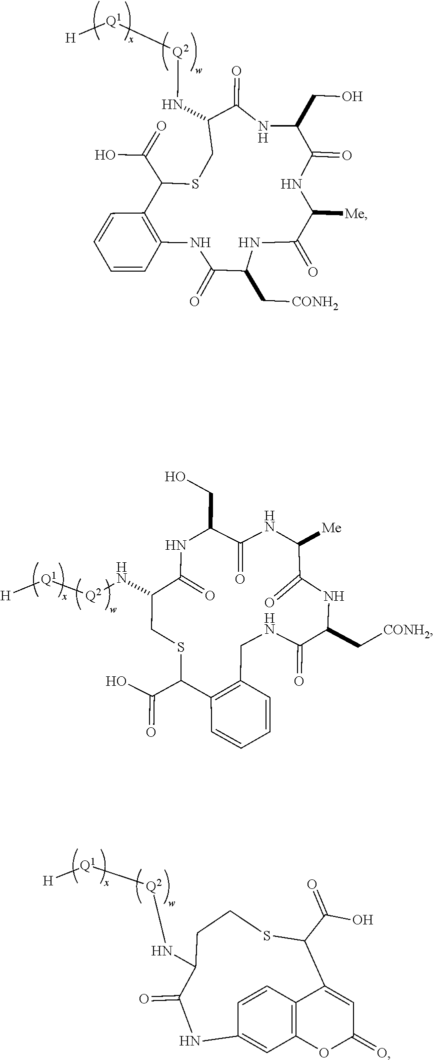

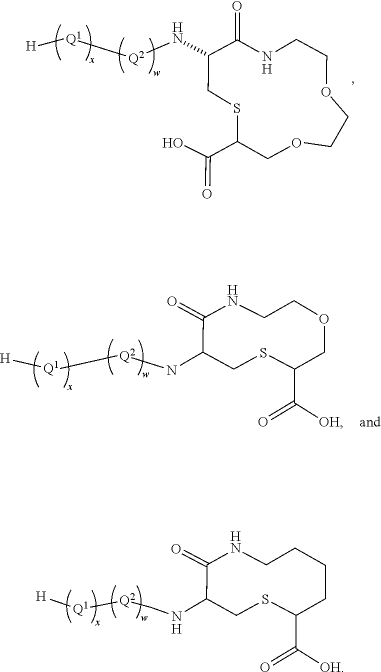

In various aspects, the thioether compound is a sactipeptide. In a further aspect, the sactipeptide has a structure represented by a formula selected from:

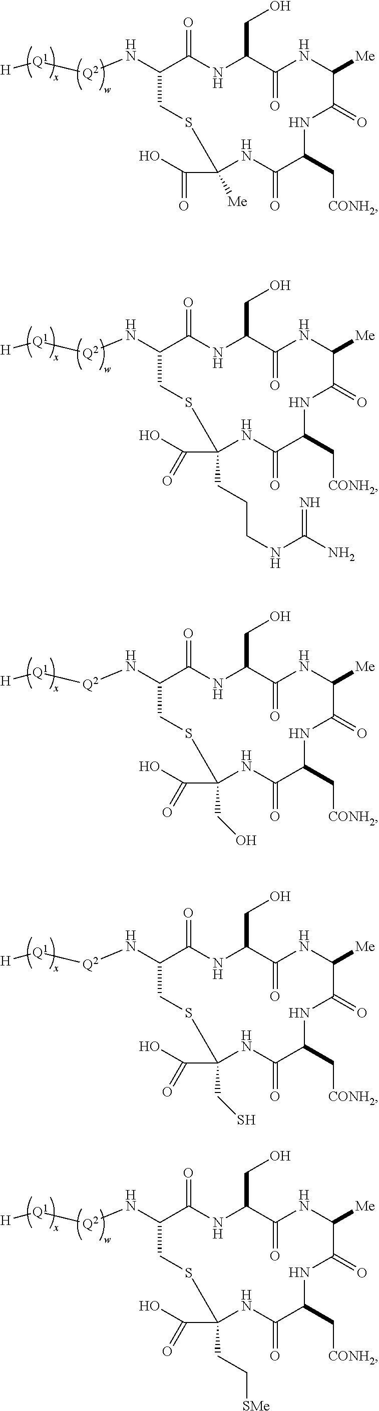

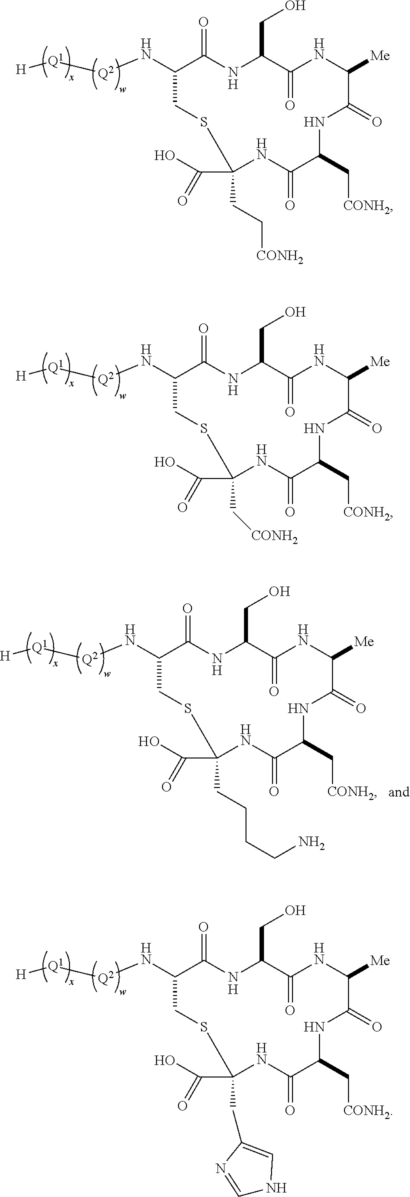

In various aspects, the thioether compound is a ranthipeptide. In a further aspect, the ranthipeptide has a structure represented by a formula selected from:

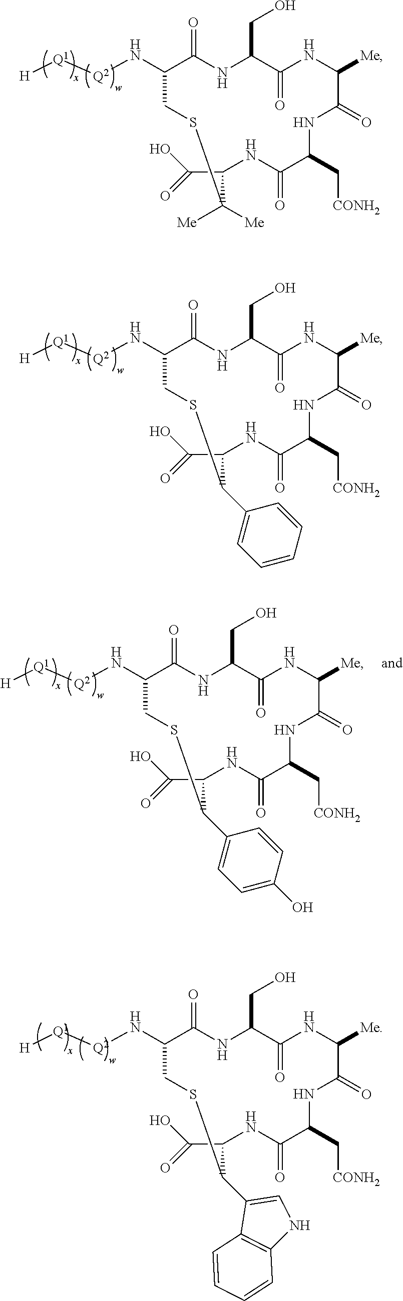

### C. METHODS OF MAKING A DNA-ENCODED LIBRARY

In one aspect, disclosed are methods of making a disclosed DNA-encoded library. As detailed herein, the sequential steps of first adding a compound as disclosed herein followed by the addition of DNA barcode with or without the use of a linker.

In various aspects, a disclosed compound can be modified with PapB, thereby forming a disclosed thioether or selenoether compound, before or after tagging with a DNA barcode. Thus, in various aspects, a disclosed compound can be modified with PapB before tagging with a DNA barcode. Alternatively, a disclosed compound can be modified with PapB after tagging with a DNA barcode.

As would be understood by one of ordinary skill in the art, the DNA barcode can be attached to the N-terminus or the C-terminus of the compound or, alternatively, can be attached to a specific side chain (e.g., cysteine, lysine, tyrosine) of the compound. Thus, in various aspects, the DNA barcode is attached to the N-terminus of the compound. In various further aspects, the DNA barcode is attached to the C-terminus of the compound. In various further aspects, the DNA barcode is attached to a side chain of the compound.

Methods of attaching a DNA barcode to a compound are well known in the art. See, e.g., Liszczak and Muir (2020) Angew. Chem. Int. Ed. Engl. 58(13): 4144-4162. Thus, in various aspects, a DNA barcode can be attached to the compound via chemical conjugation, where the barcode (typically, a modified oligonucleotide) is linked to a specific amino acid residue or functional group on the compound. Alternative techniques for attaching the compound include, but are not limited to, Edman degradation, ligase-mediated ligation, and transposase-mediated transposition. In this way, identification and tracking of the compound in complex moistures and/or during chemical reactions is facilitated.

Typically, a disclosed compound and a DNA barcode are attached through a covalent linkage between the compound and a chemically modified DNA strand by, for example, using a 5′-amine or alkyne handle on the DNA and coupling it to a carboxylic acid or azide on the compound via amide coupling or click chemistry. The barcode itself (usually a short double-stranded DNA fragment) is then enzymatically ligated to the main DNA strand using sticky-end or splint ligation.

Where the PapB modification occurs after library construction, appropriate protecting groups must be utilized for the functional groups at the -AH and R¹ positions (corresponding to residues such as, for example, cysteine, aspartic acid, and glutamic acid), which are necessary for the PapB enzymatic process. Groups such as acetamidomethyl (Acm) or tert-butoxy (OtBu) must be stable through the ligation steps but removable under mild conditions after the library is fully constructed to ensure proper function during screening.

In various aspects, a constraint for making an encoded library is that there must be compatible chemistries for adding a compound to a nucleotide or oligonucleotide sequence (e.g. DNA barcode). In various aspects, the problem of synthesis compatibility is solved by the correct choice of compatible protecting groups on the compounds, and by the correct choice of methods for deprotection to attach to the DNA barcode.

In various aspects, a linker is not required. Thus, in various aspects, the compound is directly conjugated to the DNA using a chemically modified oligonucleotide that already contains a reactive handle (e.g., a 5′-amino or 5′-alkyne-modified DNA strand). In this way, the first building block (e.g., a carboxylic acid-terminated small molecule or peptide) can be directly coupled to the DNA via amide bond formation or click chemistry, without the need for an intermediary linker.

For some architectures, and, in particular, for architectures that require solid-phase synthesis, controlled orientation of the compound, or downstream modifications, the use of a linker with specific termini for attachment of the compound, the DNA barcode, and, in some cases, for solid-phase anchoring can be advantageous. Such linkers can be particularly useful when spatial separation of the compound from the DNA is needed (e.g., to avoid steric hindrance during screening or enzymatic reactions) or when on-bead synthesis is employed.

In various aspects, a linker can be used that has suitable means for operatively linking the compound and for operatively linking the DNA barcode whose sequence codes for (defines) the compound. In various aspects, the linker has a means for attachment to a solid support, and as such allows for the synthesis to proceed in the solid phase. Thus, in various aspects the linker has a structure A′-B—C′, where A′ represents a termini adapted for reaction to operatively link a compound disclosed herein, and C′ represents a termini adapted for reaction to operatively link a nucleotide or DNA barcode. In various aspects, the termini A′ and C′ can be protected by respective blocking groups so that during operative linking reactions at one termini, the other termini is protected from reaction.

In various aspects, the linker can be subjected to a first cycle of synthesis to add a building block at one termini. The order of synthesis is not generally important insofar as one may elect to add a compound first to termini A′, or add a DNA barcode first to termini C′. A first cycle involves the steps of deprotecting the termini of the linker to which a building block is to be added and then adding the building block to the termini. Typically, the added building block contains a blocking group at its free termini, i.e., the termini that will participate in an addition of the next building block of its type. The linker molecule is then subjected to a second cycle of synthesis to add a building block at the other (second) termini. A second cycle involves the steps of deprotecting the second termini of the linker to which a building block is to be added and then adding the building block to the termini. Again, the added building block is typically blocked at its free termini.

The addition of a DNA barcode to termini C′ can be conducted either nucleotide by nucleotide to form the complete DNA barcode sequence, or the complete DNA barcode can be presynthesized, and the complete DNA barcode can be added as a block to termini C′. Insofar as the synthesis of oligonucleotides is well known in the arts, the presynthesis of oligonucleotides, and their addition to the growing nucleotide polymer in blocks is preferred because it reduces the number of manipulations in synthesizing a complex.

In various aspects, a compound or DNA barcode can be referred to as a precursor compound or precursor DNA barcode to indicate that it contains a leaving group compatible with the reaction chemistry that facilitates the precursor's operative linkage to the growing complex at the appropriate termini.

In some aspects, after the complex is formed, aliquots of the product are made, and the cycles repeated on each aliquot, with the exception that a different compound (and its corresponding DNA barcode) is added in each different aliquot.

In various aspects, the aliquots each containing a product are combined (pooled) to form a mixture of different complexes comprising a compound linked to a DNA barcode, and the mixture is divided into aliquots. In various aspects, the cycles can be repeated again on each aliquot, with different compound and DNA barcode building blocks being added to each aliquot.

In various aspects, the provided linker in step (1) is first divided into aliquots, and the cycles of step (2) are conducted on each aliquot adding a different X and corresponding Z to the linker in each different aliquot. The aliquots are then pooled as before, and the cycles of step (2) can be repeated on one or more aliquots.

In various aspects, a method for forming a disclosed library includes the steps for addition of the PCR primer binding sites to each of the complexes in the library. The method is substantially the same as above, but includes the addition of a series of nucleotides or a presynthesized primer binding sites to the linker provided in the steps above. In various aspects, all members of the library can contain the same primer binding site sequence, this sequence can be added to the A′ or C′ termini of linker prior to dividing the linker into aliquots. Thus, in various aspects, all members of the library can contain a common primer binding site which universal PCR reactions can be conducted, regardless of the species of complexes present in the library.

### 1. Libraries

In one aspect, disclosed are libraries, and methods of producing the libraries, wherein the libraries comprise a plurality of complexes, and wherein each complex comprises a DNA barcode attached to a compound having a thiol or selenol and a carboxylate, which functional groups facilitate enzymatic cross-linking using PapB. These functional groups can be embedded in a variety of different molecular frameworks, including, but not limited to, peptidomimetics and small molecule-like backbones. Specific examples of compounds that can be attached to a DNA barcode are detailed herein.

In various aspects, the compatible chemistries for sequentially adding amino acids and oligonucleotides to the growing polymers has been developed for the synthesis of an amino acid polymer in the direction of carboxy to amino terminus, and alternatively in the direction of amino to carboxy terminus. Chemistries have also been developed for the synthesis of an oligonucleotide polymer in the direction of 3′ to 5′, and alternatively in the direction of 5′ to 3′. In addition, in each of these syntheses it is preferred that the amino acid side chains (R groups) be blocked for certain amino acid residues where the R group provides an otherwise reactive termini during one of the synthesis or deblocking steps. Each type of chemistry will be described in detail herein below.

a. Compound Synthesis

For the synthesis of a compound on the linker substrate in the direction of carboxy to amino terminus, a free amino terminus on the linker is required that can be conveniently blocked and deblocked as needed. An exemplary amino terminus blocking group is a fluromethoxycarbonyl group (FMOC).

FMOC blocked amino termini are deblocked with (DBU) in dichloromethane (DCM) as is well known for polypeptide synthesis. The amino acid units are added in the form of blocked amino acids having FMOC blocked amino termini and a carboxyl terminus blocked with pentafluorophenyl ester (Opfp). The addition reaction requires the blocked amino acid, dimethylformamide (DMF) and hydroxy-benzotriazole (HOBt) as is well known for peptide synthesis. The resulting product contains an added amino acid residue with a FMOC-blocked amino terminus, ready for deblocking addition of a subsequent blocked amino acid as before.

For synthesis of a compound on the linker substrate in the direction of amino to carboxy terminus, a free carboxy terminus on the linker is required that can be conveniently blocked and deblocked as needed. An exemplary carboxy terminus blocking group is the Opfp ester described before.

Briefly, a carboxy terminus on the linker is produced by reacting a linker with a free amino terminus with succinamide in HOBE and a proton catalyst. Thereafter, the terminus can be modified by reaction with pentafluorophenol in dichlorocarbodiimide (DCC) and ethanol acetate to form an Opfp ester at the free carboxy terminus. The Opfp ester is blocked linker terminus is available for addition reaction with a FMOC-, Opfp-blocked amino acid as before, but with the amino acid adding to the linker in the reverse direction. The resulting product contains an added amino acid residue with an Opfp-blocked terminus, ready to repeat the addition with a subsequent blocked amino acid.

b. DNA Barcode Synthesis

DNA barcodes are commercially available or can be synthesized by a variety of chemistries well-known in the art. See, e.g., “Oligonucleotide Synthesis: A Practical Approach,” ed. M. J. Gait, JRL Press, New York, N.Y. (1990).

### D. METHODS OF SCREENING USING A DNA-ENCODED LIBRARY

In one aspect, disclosed are methods of screening using a DNA-encoded library, the method comprising: (a) contacting complexes, wherein each complex comprises one or more compounds and each compound has a unique DNA barcode, with PapB under conditions sufficient to allow the compound to form a thioether or selenoether crosslink, thereby forming a complex comprising a cross-linked compound, wherein the one or more compounds has a structure represented by a formula:

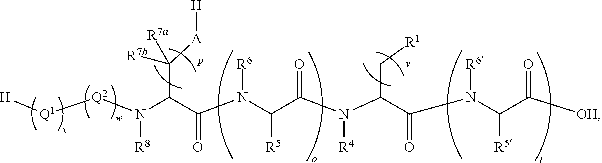

wherein o is 0, 1, 2, 3, 4, 5, 6, 7, 8, or 9; wherein p is 1 or 2; wherein t is an integer from 0 to 500; wherein v is 1, 2, 3, 4, or 5; wherein w is 0 or 1; wherein x is 0 or 1; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

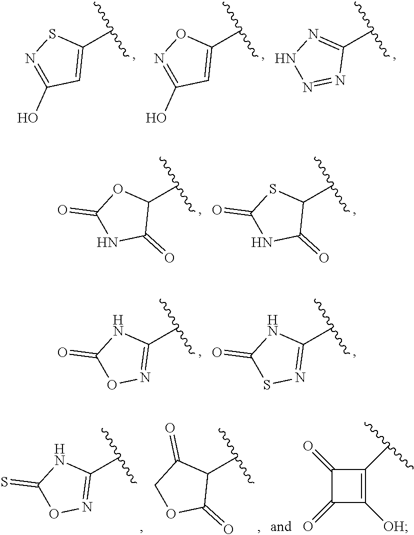

wherein R⁴ is selected from hydrogen and methyl; wherein each occurrence of R⁵ and R^{5′} when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, (b) incubating complexes comprising the cross-linked compound with one or more biological targets; (c) isolating the complexes comprising the cross-linked compound bound to the biological target; and (d) obtaining the sequence of the unique DNA barcode of each of the one or more complexes comprising the cross-linked compound bound to the biological target, wherein the sequence of the unique DNA barcode identifies the compound bound to the biological target.

In one aspect, disclosed are methods of screening using a DNA-encoded library, the method comprising: (a) contacting complexes, wherein each complex comprises one or more compounds and each compound has a unique DNA barcode, with PapB under conditions sufficient to allow the compound to form a thioether or selenoether crosslink, thereby forming a complex comprising a cross-linked compound, wherein the one or more compounds has a structure represented by a formula:

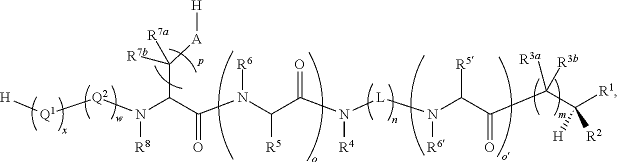

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein x is 0 or 1; wherein A is S or Se; wherein A is S or Se; wherein L, when present, is selected from C2-C4 alkyl, —(C1-C4 alkyl)(OCH₂CH₂)_{q}, and a structure selected from:

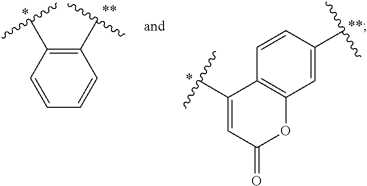

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

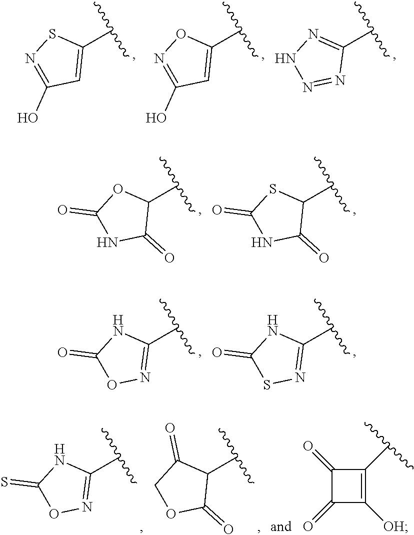

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, (b) incubating complexes comprising the cross-linked compound with one or more biological targets; (c) isolating the complexes comprising the cross-linked compound bound to the biological target; and (d) obtaining the sequence of the unique DNA barcode of each of the one or more complexes comprising the cross-linked compound bound to the biological target, wherein the sequence of the unique DNA barcode identifies the compound bound to the biological target.

In various aspects, the method of screening can first comprise obtaining one or more compounds disclosed herein. In various aspects, the obtaining one or more compounds comprises obtaining one or more compounds that comprise a DNA barcode. In various aspects, the compounds can be contacted with PapB before the compounds are conjugated to a DNA barcode. In various aspects, the compounds can be contacted with PapB after the compounds are conjugated to a DNA barcode.

In various aspects, the disclosed methods of screening can begin with the step of incubating complexes comprising the cross-linked compound with one or more biological targets. When the methods being with the incubating step, this means the steps of obtaining a compound and contacting the compound with PapB occur prior to the screening.

As detailed herein, the disclosed libraries provides a repertoire of chemical diversity such that each compound (e.g., compound, thioether compound, selenoether compound) is linked to a DNA barcode or genetic tag that facilitates identification of the compound. The disclosed screening methods can be used to identify a compound that participates in a preselected binding interaction between the compound and a biological target. The compound to be identified is represented by one of the members of a library of this invention, and the method comprises the following steps: (1) Incubating complexes comprising one or more of the disclosed cross-linked compounds comprising a DNA barcode with one or more biological targets; (2) Isolating the complexes comprising the cross-linked compound bound to the biological target; and (3) Obtaining the sequence of the DNA barcode of each of the one or more complexes comprising the cross-linked compound bound to the biological target, wherein the sequence of the DNA barcode identifies the compound bound to the biological target.

In various aspects, a biological target can be any of a variety of molecules that bind selectively to another molecule, including antibodies to antigens, lectins to oligosaccharides, receptors to ligands, enzymes to substrates and the like mediators of molecular interactions. In various aspects, the one or more biological targets are from a specific tissue type. In various aspects, the one or more biological targets are from a specific disease pathway.

In various aspects, macrocyclic hits from phage or mRNA display can be utilized as initial scaffolds for follow-up screening via DEL. Systematic substitution at multiple positions could then be accomplished using unnatural amino acids (UAAs) not accessible in the original screen. In this way, enzymatic macrocyclization can be applied in the context of a chemically expanded DEL to yield a library of conformationally constrained variants with potentially improved binding, selectivity, and/or other properties.

It is contemplated that the disclosed methods can be used in connect in with the compounds disclosed herein.

1. Contacting Complexes with PAPB

In various aspects, the methods comprise contacting complexes, wherein each complex comprises one or more compounds and each compound has a unique DNA barcode, with PapB under conditions sufficient to allow the compound to form a thioether or selenoether crosslink, thereby forming a complex comprising a cross-linked compound.

In various aspects, the unique DNA barcode can attached to the compounds disclosed here. The unique DNA barcode can be attached to the N-terminus, C-terminus, or specific side chains (e.g., cysteine, lysine, tyrosine) of the compound.

In various aspects, the contacting of the compound having a DNA barcode with PapB can occur prior to the method of screening. Thus, in various aspects, the method of screening can begin with the incubating step described below.

2. Incubating Complexes with Biological Target

In various aspects, after forming a complex comprising a cross-linked compound, the methods comprise incubating the complex comprising the cross-linked compound with one or more biological targets.

In various aspects, incubating the complex comprising the cross-linked compound with one or more biological targets can occur for any length of time that allows for binding of the cross-linked compound to a biological target under natural conditions. For example, some cross-linked compounds will not bind to a biological target and therefore, no matter how long the incubation lasts, binding will not occur. Thus, in various aspects, the length of time for the incubating step can be based on the amount of time that the biological target would bind to one of its known binding partners or the amount of time that the cross-linked compound would bind to one of its known binding partners. In various aspects, the length of time can be 10, 15, 20, 25, 30, 35, 40, 45, 50, 55, or 60 min. In various aspects, the length of time can be 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 hrs. In various aspects, the length of time can be 12 hrs to 24 hrs or 24 hrs to 48 hrs.

In various aspects, binding conditions are those conditions compatible with the known natural binding function of the biological target. Those compatible conditions can be buffer, pH, and temperature conditions that maintain the biological activity of the biological target, thereby maintaining the ability of the biological target to participate in its preselected binding interaction. In various aspects, the incubation (or binding) conditions include an aqueous, physiologic solution of pH and ionic strength normally associated with the biological target.

In various aspects, complexes comprising the cross-linked compound bound to the biological target can be referred to as a stable product of the interaction. In various aspects, a stable product is maintained over sufficient time that the complex comprising the cross-linked compound bound to the biological target can be isolated from the rest of the members of the library without the complex becoming significantly disassociated.

### 3. Isolating Complexes Bound to Biological Target

In various aspects, after forming complexes comprising the cross-linked compound bound to the biological target, the methods can comprise isolating the complexes comprising the cross-linked compound bound to the biological target.

In various aspects, the biological target or the complex comprising the compound and DNA barcode can be immobilized on a solid support. Thus, in various aspects, during the step of contacting complexes, wherein each complex comprises one or more compounds and each compound has a unique DNA barcode, with PapB, and/or the step of incubating the complexes comprising the cross-linked compound with one or more biological targets, a solid support can be present.

In various aspects, the biological target is bound to or immobilized on a solid support. In various aspects, the biological target can be immobilized on the solid support on the N-terminal end, C-terminal end, or anywhere in between that does not alter its binding ability. In various aspects, the compound having a DNA barcode is bound to or immobilized on a solid support. In various aspects, when the compound having a DNA barcode is bound to the solid support, the immobilization occurs at a linker between the compound and the DNA barcode. Any known techniques for coupling or conjugating a nucleic acid or amino acid to a solid support can be used.

In various aspects, the solid support comprises biotin, protein A or magnetic beads. In a further aspect, the solid support comprises biotin. In a still further aspect, the solid support comprises protein A. In yet a further aspect, the solid support comprises or magnetic beads. In various aspects, the solid support can be of any solid material, for example a plastic, such as polystyrene or polycarbonate. In various aspects, the solid support can be a multi-compartment vessel for screening multiple candidates at once. In various aspects, the solid support can be, but is not limited to, paramagnetic particles (PMPs), latex particles, paramagnetic latex particles, membranes, polystyrene microbeads, glass beads, beaded gel resins, polystyrene resins, PEG-based gel resins, and magnetic particles. The use of solid supports on which to operatively link proteinaceous molecules is generally well known in the art. Useful solid support matrices are well known in the art and include cross-linked dextran such as that available under the tradename SEPHADEX from Pharmacia Fine Chemicals (Piscataway, N.J.); agarose, borosilicate, polystyrene or latex beads about 1 micron to about 5 millimeters in diameter, polyvinyl chloride, polystyrene, cross-linked polyacrylamide, nitrocellulose or nylon-based webs such as sheets, strips, paddles, plates microtiter plate wells and the like insoluble matrices.

In various aspects, isolating the complexes comprising the cross-linked compound bound to the biological target comprises washing unbound complexes away from the cross-linked compound bound to the biological target. In various aspects, if the complex is what is bound to the solid support, then isolating the complexes comprising the cross-linked compound bound to the biological target comprises washing unbound biological target away from the cross-linked compound bound to the biological target.

In various aspects, isolating comprises any separation means that is selective for the complex (or the biological target), thereby isolating that complex that has bound to the biological target. There are a variety of separation means, depending on the biological target.

In various aspects, an immobilized antibody immunospecific for the biological target can be provided as a solid phase-affixed antibody after the complex has bound to the biological target. The immobilized antibody immunoreacts with the biological target present in the complex of cross-linked compound bound to the biological target to form an antibody-biological target immunoreaction complex. Thereafter, by separation of the solid phase, the immunoreaction complex, and therefore anything bound to the immunoreaction complex (e.g. cross-linked compound having a DNA barcode), is separated from the unbound cross-linked compound having a DNA barcode.

In various aspects, any binding pair commonly used for isolating or separating can be used. In various aspects, a binding means can be operatively linked to the biological target to facilitate its retrieval after incubating with the cross-linked compound. Exemplary binding means are one of the following high affinity pairs: biotin-avidin, protein A-Fc receptor, ferritin-magnetic beads, and the like. Thus, the biological target is operatively linked (conjugated) to biotin, protein A, ferritin and the like binding means, and the complex comprising the cross-linked compound bound to the biological target is isolated by the use of the corresponding binding partner in the solid phase, e.g., solid-phase avidin, solid-phase Fc receptor, solid phase magnetic beads and the like.

### 4. Obtaining the Sequence of the DNA Barcode

In various aspects, the methods comprise obtaining the sequence of the unique DNA barcode of each of the one or more complexes comprising the cross-linked compound bound to the biological target, wherein the sequence of the unique DNA barcode identifies the compound bound to the biological target

In various aspects, the nucleotide sequence of the DNA barcode present in the isolated complexes is determined to identify the compound (e.g., compound, thioether compound, or selenoether compound) that was present in the compound:biological target interaction.

In various aspects, obtaining the sequence of the unique DNA barcode comprises amplifying and/or sequencing the unique DNA barcode. In various aspects, the amplifying comprises polymerase chain reaction (PCR).

Although conventional DNA sequencing methods are readily available and useful for this determination, the amount and quality of isolated complex may require additional manipulations prior to a sequencing reaction.

In various aspects, where the amount of DNA barcode is low, it is preferred to increase the amount of the DNA barcode by PCR using PCR primers directed to the primer binding sites present in the DNA barcode.

In various aspects, the quality of the isolated complex may be such that multiple complexes can be co-isolated by virtue of similar capacities for binding to the biological target. In cases where more than one complex is isolated, the different isolated complexes must be separated prior to sequencing of the DNA barcode.

Thus, in various aspects, the different DNA barcodes of the isolated complexes can be cloned into separate sequencing vectors prior to determining their sequence by DNA sequencing methods. In various aspects, this can be accomplished by amplifying all of the different DNA barcodes by PCR as described herein, and then using the unique restriction endonuclease sites on the amplified product to directionally clone the amplified fragments into sequencing vectors. The cloning and sequencing of the amplified fragments then is a routine procedure that can be carried out by any of a number of molecular biological methods known in the art.

In various aspects, PCR amplified products derived from a population of isolated complexes can be used as a hybridization probe to selectively enrich the quality of the isolated complexes. For example, using the hybridization probes, one can isolate members of the library by hybridization to form an enriched library containing only complexes that have sequences that hybridize to the above hybridization probes. In a second screening reaction under different binding conditions, for example, higher stringency binding conditions, one can isolate the complexes that bind most tightly with the biological target.

In various aspects, for determining the nucleotide sequence of the DNA barcode in the isolated complex, polymerase chain reaction (PCR) can be used.

In various aspects, where the genetic material to be assayed is in the form of double stranded DNA, it is usually first denatured, typically by melting, into single strands. The nucleic acid can be subjected to a PCR reaction by treating (contacting) the sample with a PCR primer pair, each member of the pair having a preselected nucleotide sequence. The PCR primer pair is capable of initiating primer extension reactions by hybridizing to the PCR primer binding site on nucleotide sequences of the DNA barcode, preferably at least about 10 nucleotides in length, more preferably at least about 20 nucleotides in length and most preferably 17 nucleotides in length. The first primer of a PCR primer pair is sometimes referred to herein as the “anti-sense primer” because it hybridizes to a non-coding or anti-sense strand of a nucleic acid, i.e., a strand complementary to a coding strand. The second primer of a PCR primer pair is sometimes referred to herein as the “sense primer” because it hybridizes to the coding or sense strand of a nucleic acid.

In various aspects, the PCR reaction can be performed by mixing the PCR primer pair, preferably a predetermined amount thereof, with the nucleic acids of the sample, preferably a predetermined amount thereof, in a PCR buffer to form a PCR reaction admixture. The admixture is thermocycled for a number of cycles, which is typically predetermined, sufficient for the formation of a PCR reaction product, thereby enriching the sample to be assayed for the DNA barcode in the isolated complex.

PCR is typically carried out by thermocycling, i.e., repeatedly increasing and decreasing the temperature of a PCR reaction admixture within a temperature range whose lower limit is about 30 °C to about 55 °C and whose upper limit is about 90 °C to about 100 °C. The increasing and decreasing can be continuous, or can be phasic with time periods of relative temperature stability at each of temperatures favoring polynucleotide synthesis, denaturation, and hybridization.

In various aspects, a plurality of first primer and/or a plurality of second primers can be used in each amplification, e.g., one species of first primer can be paired with a number of different second primers to form several different primer pairs. Alternatively, an individual pair of first and second primers can be used. In any case, the amplification products of amplifications using the same or different combinations of first and second primers can be combined for assaying for mutations.

The PCR reaction can be performed using any suitable method. Generally, it occurs in a buffered aqueous solution, i.e., a PCR buffer, preferably at a pH of 7-9, most preferably about 8. Preferably, a molar excess of the primer is admixed to the buffer containing the template strand. A large molar excess is preferred to improve the efficiency of the process.

In various aspects, the PCR buffer can also contain the deoxyribonucleotide triphosphates (polynucleotide synthesis substrates) dATP, dCTP, dGTP, and dTTP and a polymerase, typically thermostable, all in adequate amounts for primer extension (polynucleotide synthesis) reaction. The resulting solution (PCR admixture) is heated to about 90 °C to 100 °C for about 1 to 10 minutes, preferably from 1 to 4 minutes. After this heating period the solution is allowed to cool to 54 °C, which is preferable for primer hybridization. The synthesis reaction may occur at from room temperature up to a temperature above which the polymerase (inducing agent) no longer functions efficiently. Thus, for example, if DNA polymerase is used as inducing agent, the temperature is generally no greater than about 40 °C. The thermocycling is repeated until the desired amount of PCR product is produced. An exemplary PCR buffer comprises the following: 50 mM KCl; 10 mM Tris-HCl at pH 8.3; 1.5 mM MgCl₂; 0.001% (wt/vol) gelatin, 200 M dATP; 200 M dTTP; 200 M dCTP; 200 M dGTP; and 2.5 units Thermus aquaticus (Taq) DNA polymerase I (U.S. Pat. No. 4,889,818) per 100 microliters (l) of buffer.

In various aspects, the inducing agent may be any compound or system that will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, including heat-stable enzymes, which will facilitate combination of the nucleotides in the proper manner to form the primer extension products which are complementary to each nucleic acid strand. Generally, the synthesis can be initiated at the 3′ end of each primer and proceed in the 5′ direction along the template strand, until synthesis terminates, producing molecules of different lengths. There can be inducing agents, however, which initiate synthesis at the 5′ end and proceed in the above direction, using the same process as described above.

In various aspects, the inducing agent also can be a compound or system which will function to accomplish the synthesis of RNA primer extension products, including enzymes. In preferred embodiments, the inducing agent may be a DNA-dependent RNA polymerase such as T7 RNA polymerase, T3 RNA polymerase or SP6 RNA polymerase. These polymerases produce a complementary RNA polynucleotide. The high turn-over rate of the RNA polymerase amplifies the starting polynucleotide as has been described by Chamberlin et al., The Enzymes, ed. P. Boyer, pp. 87-108, Academic Press, New York (1982). Amplification systems based on transcription have been described by Gingeras et al., in PCR Protocols, A Guide to Methods and Applications, pp. 245-252, Innis et al., eds, Academic Press, Inc., San Diego, Calif (1990).

In various aspects, tf the inducing agent is a DNA-dependent RNA polymerase and, therefore incorporates ribonucleotide triphosphates, sufficient amounts of ATP, CTP, GTP and UTP are admixed to the primer extension reaction admixture and the resulting solution is treated as described above.

In various aspects, the newly synthesized strand and its complementary nucleic acid strand form a double-stranded molecule which can be used in the succeeding steps of the process.

PCR amplification methods are described in detail in U.S. Pat. Nos. 4,683,192, 4,683,202, 4,800,159, and 4,965,188, and at least in several texts including PCR Technology: Principles and Applications for DNA Amplification, H. Erlich, ed., Stockton Press, New York (1989); and PCR Protocols: A Guide to Methods and Applications, Innis et al., eds., Academic Press, San Diego, Calif (1990).

The term “polynucleotide” as used herein in reference to primers, probes, and nucleic acid fragments or segments to be synthesized by primer extension is defined as a molecule comprised of two or more deoxyribonucleotides or ribonucleotides, preferably more than three. Its exact size will depend on many factors, which in turn depends on the ultimate conditions of use.

The term “primer” as used herein refers to a polynucleotide whether purified from a nucleic acid restriction digest or produced synthetically, which is capable of acting as a point of initiation of nucleic acid synthesis when placed under conditions in which synthesis of a primer extension product which is complementary to a nucleic acid strand is induced, i.e., in the presence of nucleotides and an agent for polymerization such as DNA polymerase, reverse transcriptase and the like, and at a suitable temperature and pH. The primer is preferably single stranded for maximum efficiency, but may alternatively be in double stranded form. If double stranded, the primer is first treated to separate it from its complementary strand before being used to prepare extension products. In various aspects, the primer is a polydeoxyribonucleotide. In various aspects, the primer must be sufficiently long to prime the synthesis of extension products in the presence of the agents for polymerization. In various aspects, the exact lengths of the primers will depend on many factors, including temperature and the source of primer. For example, depending on the complexity of the target sequence, a polynucleotide primer typically contains 15 to 25 or more nucleotides, although it can contain fewer nucleotides. Short primer molecules generally require cooler temperatures to form sufficiently stable hybrid complexes with template.

In various aspects, the primers used herein are selected to be “substantially” complementary to the different strands of each specific sequence to be synthesized or amplified. This means that the primer must be sufficiently complementary to non-randomly hybridize with its respective template strand. Therefore, the primer sequence may or may not reflect the exact sequence of the template. For example, a non-complementary nucleotide fragment can be attached to the 5′ end of the primer, with the remainder of the primer sequence being substantially complementary to the strand. Such non-complementary fragments typically code for an endonuclease restriction site. Alternatively, non-complementary bases or longer sequences can be interspersed into the primer, provided the primer sequence has sufficient complementarity with the sequence of the strand to be synthesized or amplified to non-randomly hybridize therewith and thereby form an extension product under polynucleotide synthesizing conditions.

In various aspects, primers of the present invention can also contain a DNA-dependent RNA polymerase promoter sequence or its complement. See for example, Krieg et al., Nucl. Acids Res., 12:7057-70 (1984); Studier et al., J. Mol. Biol., 189:113-130 (1986); and Molecular Cloning: A Laboratory Manual, Second Edition, Maniatis et al., eds., Cold Spring Harbor, N.Y. (1989).

In various aspects, when a primer containing a DNA-dependent RNA polymerase promoter is used, the primer is hybridized to the polynucleotide strand to be amplified and the second polynucleotide strand of the DNA-dependent RNA polymerase promoter is completed using an inducing agent such as E. coli DNA polymerase I, or the Klenow fragment of E. coli DNA polymerase. The starting polynucleotide is amplified by alternating between the production of an RNA polynucleotide and DNA polynucleotide.

In various aspects, primers can also contain a template sequence or replication initiation site for a RNA-directed RNA polymerase. Typical RNA-directed RNA polymerase include the QB replicase described by Lizardi et al., Biotechnology, 6:1197-1202 (1988). RNA-directed polymerases produce large numbers of RNA strands from a small number of template RNA strands that contain a template sequence or replication initiation site. These polymerases typically give a one million-fold amplification of the template strand as has been described by Kramer et al., J. Mol. Biol., 89:719-736 (1974).

In various aspects, the polynucleotide primers can be prepared using any suitable method, such as, for example, the phosphotriester or phosphodiester methods see Narang et al., Meth. Enzymol., 68:90, (1979); U.S. Pat. Nos. 4,356,270, 4,458,066, 4,416,988, 4,293,652; and Brown et al., Meth. Enzymol., 68:109, (1979).

If the nucleic acid sample is to be enriched for the DNA barcode in the isolated complex by PCR amplification, two primers, i.e., a PCR primer pair, must be used for each coding strand of nucleic acid to be amplified. The first primer becomes part of the non-coding (anti-sense or minus or complementary) strand and hybridizes to a nucleotide sequence on the plus or coding strand. Second primers become part of the coding (sense or plus) strand and hybridize to a nucleotide sequence on the minus or non-coding strand. One or both of the first and second primers can contain a nucleotide sequence defining an endonuclease recognition site as described herein. The site can be heterologous to the DNA barcode being amplified.

In various aspects, the present invention utilizes a set of polynucleotides that form primers having a priming region located at the 3′-terminus of the primer. The priming region is typically the 3′-most (3′-terminal) 15 to 30 nucleotide bases. The 3′-terminal priming portion of each primer is capable of acting as a primer to catalyze nucleic acid synthesis, i.e., initiate a primer extension reaction off its 3′ terminus. One or both of the primers can additionally contain a 5′-terminal (5′-most) non-priming portion, i.e., a region that does not participate in hybridization to the preferred template.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A deoxyribonucleic acid- (DNA-) encoded library comprising a plurality of complexes, wherein each complex comprises:
(a) one or more DNA barcodes; and
(b) one or more compounds having a structure represented by a formula: 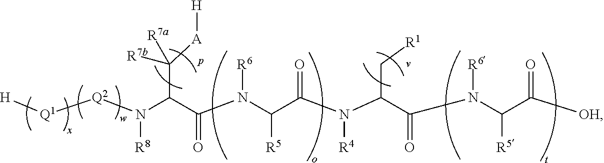
wherein o is 0, 1, 2, 3, 4, 5, 6, 7, 8, or 9;
wherein p is 1 or 2;
wherein t is an integer from 0 to 500;
wherein v is 1, 2, 3, 4, or 5;
wherein w is 0 or 1;
wherein x is 0 or 1;
wherein A is S or Se;
wherein Q¹ is a leader sequence;
wherein Q² is a cleavable moiety;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 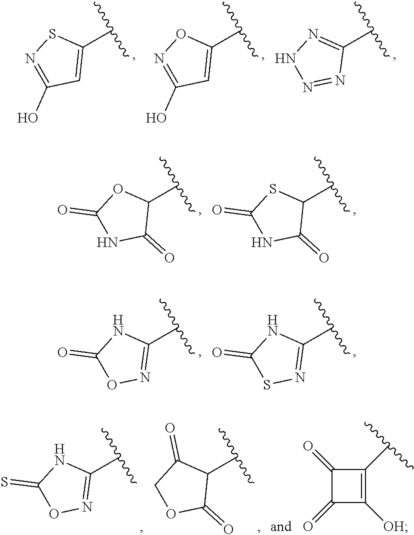
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl; and
wherein R⁸ is selected from hydrogen and methyl,
wherein at least one of the DNA barcodes is attached to at least one of the compounds.

2. The DNA-encoded library of claim 1, wherein each DNA barcode is unique to a single compound.

3. The DNA-encoded library of claim 1, wherein each DNA barcode is 5 to 15 nucleotides in length.

4. The DNA-encoded library of claim 1, wherein at least one of the DNA barcodes attached to at least one of the compounds comprises at least one of the DNA barcodes conjugated or linked to at least one of the compounds.

5. The DNA-encoded library of claim 1, wherein the complex further comprises a linker between at least one of the DNA barcodes and at least one of the compounds.

6. The DNA-encoded library of claim 1, wherein the DNA barcode is single stranded or double stranded.

7. The DNA-encoded library of claim 1, wherein R¹ is —CO₂H or a structure:

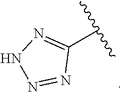 [SMILES: *c1nn[nH]n1]

8. The DNA-encoded library of claim 1, wherein the cleavable moiety is —CO₂—(C4-C8 alkylene)-OC(O)—.

9. The DNA-encoded library of claim 1, wherein the cleavable moiety is a protease recognition sequence.

10. The DNA-encoded library of claim 1, wherein the one or more compounds have a structure represented by a formula:

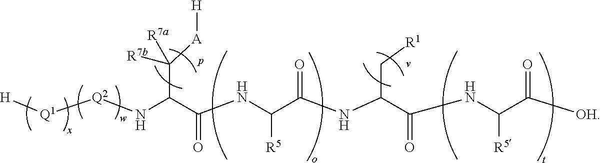

11. The DNA-encoded library of claim 1, wherein the one or more compounds have a structure represented by a formula:

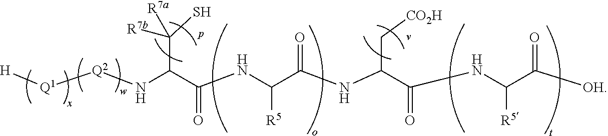

12. A deoxyribonucleic acid- (DNA-) encoded library comprising a plurality of complexes, wherein each complex comprises:
(a) one or more DNA barcodes; and
(b) one or more compounds having a structure represented by a formula: 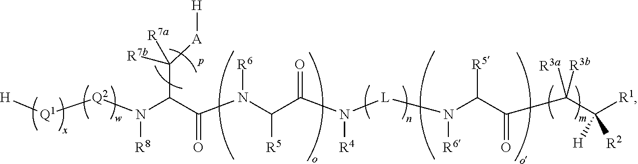
wherein m is 0, 1, 2, 3, or 4;
wherein n is 0 or 1;
wherein each of o and o′ is independently 0, 1, 2, 3, 4, 5, 6, 7, 8, or 9;
wherein p is 1 or 2;
wherein w is 0 or 1;
wherein q is 0 or 1;
wherein x is 0 or 1;
wherein A is S or Se;
wherein L, when present, is selected from C2-C4 alkyl, —(C1-C4 alkyl)(OCH₂CH₂)_{q}, and a structure selected from: 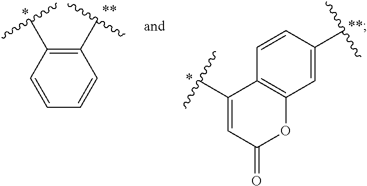
wherein q is 1, 2, 3, or 4;
wherein Q¹ is a leader sequence;
wherein Q² is a cleavable moiety;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 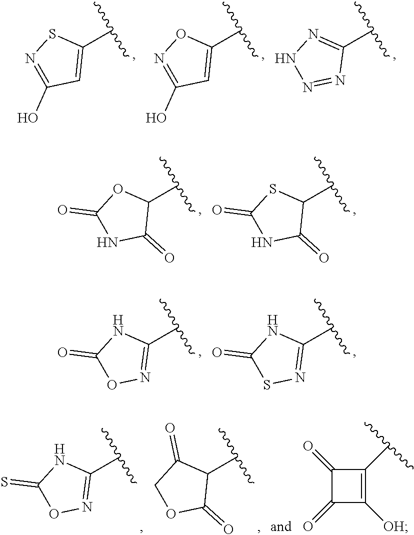
wherein R² is a residue of a side chain of amino acid, provided that the amino acid is not isoleucine or threonine;
wherein each of R^{3a} and R^{3b}, when present, is independently selected from C2-C5 alkynyl, C1-C5 azido, and a residue of a side chain of an amino acid;
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl, and
wherein R⁸ is selected from hydrogen and methyl,
wherein at least one of the DNA barcodes is attached to at least one of the compounds.

13. The DNA-encoded library of claim 21, wherein each DNA barcode is unique to a single compound.

14. The DNA-encoded library of claim 21, wherein each DNA barcode is 5 to 15 nucleotides in length.

15. The DNA-encoded library of claim 21, wherein at least one of the DNA barcodes attached to at least one of the compounds comprises at least one of the DNA barcodes conjugated or linked to at least one of the compounds.

16. The DNA-encoded library of claim 21, wherein the complex further comprises a linker between at least one of the DNA barcodes and at least one of the compounds.

17. A deoxyribonucleic acid- (DNA-) encoded library comprising a plurality of complexes, wherein each complex comprises:
(a) one or more DNA barcodes; and
(b) one or more thioether or selenoether compounds having a structure represented by a formula: 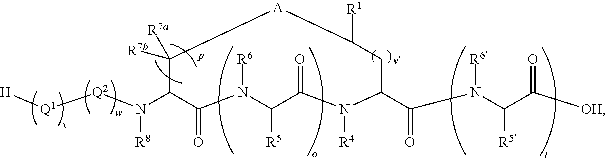
wherein o is 0, 1, 2, 3, 4, 5, 6, 7, 8, or 9;
wherein p is 1 or 2;
wherein t is an integer from 0 to 500;
wherein v′ is 0, 1, 2, or 3;
wherein w is 0 or 1;
wherein x is 0 or 1;
wherein A is S or Se;
wherein Q¹ is a leader sequence;
wherein Q² is a cleavable moiety;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 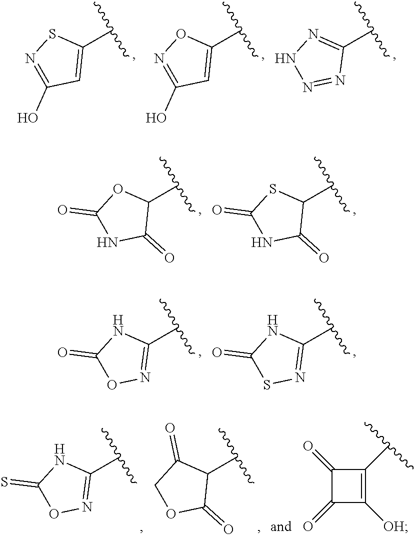
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl; and
wherein R⁸ is selected from hydrogen and methyl,
wherein at least one of the DNA barcodes is attached to at least one of the compounds.

18. A deoxyribonucleic acid- (DNA-) encoded library comprising a plurality of complexes, wherein each complex comprises:
(a) one or more DNA barcodes; and
(b) one or more thioether or selenoether compounds having a structure represented by a formula: 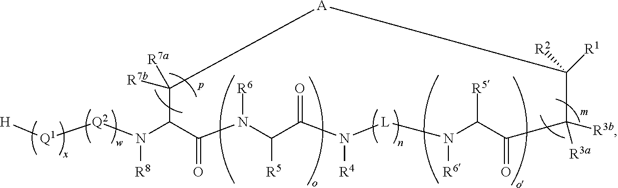
wherein m is 0, 1, 2, 3, or 4;
wherein n is 0 or 1;
wherein each of o and o′ is independently 0, 1, 2, 3, 4, 5, 6, 7, 8, or 9;
wherein p is 1 or 2;
wherein w is 0 or 1;
wherein x is 0 or 1;
wherein A is S or Se;
wherein L, when present, is selected from C2-C4 alkyl, —(C1-C4 alkyl)(OCH₂CH₂)_{q}, and a structure selected from: 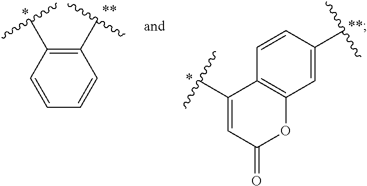
wherein q is 1, 2, 3, or 4;
wherein Q¹ is a leader sequence;
wherein Q² is a cleavable moiety;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 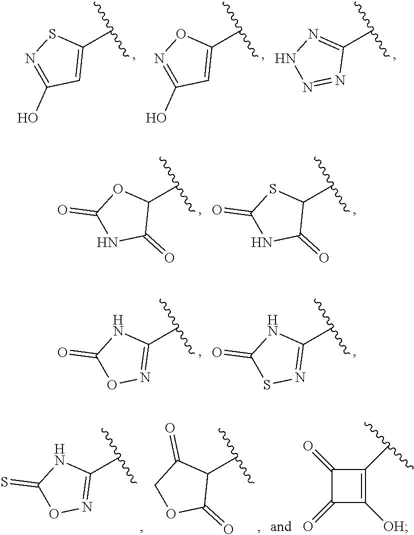
wherein R² is a residue of a side chain of amino acid, provided that the amino acid is not isoleucine or threonine;
wherein each of R^{3a} and R^{3b}, when present, is independently selected from C2-C5 alkynyl, C1-C5 azido, and a residue of a side chain of an amino acid;
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; and
wherein each of R^{7a} and R^{7b}, when present, is independently selected from hydrogen and C1-C4 alkyl,
wherein at least one of the DNA barcodes is attached to at least one of the compounds.

19. A method of screening using a DNA-encoded library, the method comprising:
(a) incubating one or more compounds with a unique DNA barcode under conditions for attachment of each of the one or more compounds to the unique DNA barcode, whereby complexes comprising the unique DNA barcode and a compound are formed, wherein the one or more compounds has a structure represented by a formula: 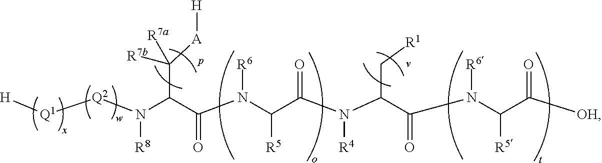
wherein o is 0, 1, 2, 3, 4, 5, 6, 7, 8, or 9;
wherein p is 1 or 2;
wherein t is an integer from 0 to 500;
wherein v is 1, 2, 3, 4, or 5;
wherein w is 0 or 1;
wherein x is 0 or 1;
wherein A is S or Se;
wherein Q¹ is a leader sequence;
wherein Q² is a cleavable moiety;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 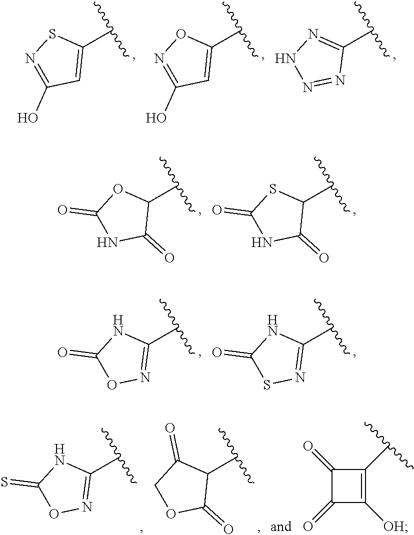
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; and
wherein each of R^{7a} and R^{7b}, when present, is independently selected from hydrogen and C1-C4 alkyl; and
wherein R⁸ is selected from hydrogen and methyl;
(b) contacting the complexes with PapB under conditions sufficient to allow the compound to form a thioether or selenoether crosslink, thereby forming a complex comprising a cross-linked compound;
(c) incubating complexes comprising the cross-linked compound with one or more biological targets;
(d) isolating the complexes comprising the cross-linked compound bound to the biological target; and
(e) obtaining the sequence of the unique DNA barcode of each of the one or more complexes comprising the cross-linked compound bound to the biological target,
wherein the sequence of the unique DNA barcode identifies the compound bound to the biological target.

20. A method of screening using a DNA-encoded library, the method comprising:
(a) incubating one or more compounds with a unique DNA barcode under conditions for attachment of each of the one or more compounds to the unique DNA barcode, whereby complexes comprising the unique DNA barcode and a compound are formed, wherein the one or more compounds has a structure represented by a formula: 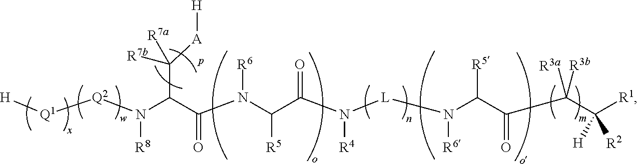
wherein m is 0, 1, 2, 3, or 4;
wherein n is 0 or 1;
wherein each of o and o′ is independently 0, 1, 2, 3, 4, 5, 6, 7, 8, or 9;
wherein p is 1 or 2;
wherein w is 0 or 1;
wherein x is 0 or 1;
wherein A is S or Se;
wherein L, when present, is selected from C2-C4 alkyl, —(C1-C4 alkyl)(OCH₂CH₂)_{q}, and a structure selected from: 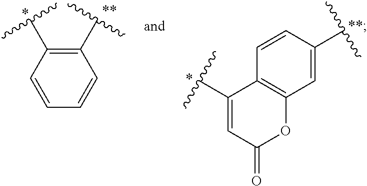
wherein q is 1, 2, 3, or 4;
wherein Q¹ is a leader sequence;
wherein Q² is a cleavable moiety;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 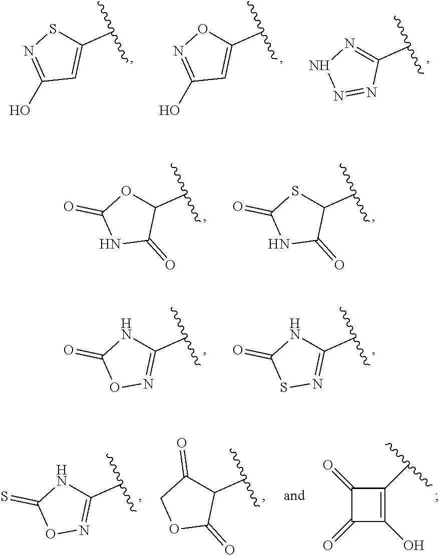
wherein R² is a residue of a side chain of amino acid, provided that the amino acid is not isoleucine or threonine;
wherein each of R^{3a} and R^{3b}, when present, is independently selected from C2-C5 alkynyl, C1-C5 azido, and a residue of a side chain of an amino acid;
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; and
wherein each of R^{7a} and R^{7b}, when present, is independently selected from hydrogen and C1-C4 alkyl;
(b) contacting the complexes with PapB under conditions sufficient to allow the compound to form a thioether or selenoether crosslink, thereby forming a complex comprising a cross-linked compound;
(c) incubating complexes comprising the cross-linked compound with one or more biological targets;
(d) isolating the complexes comprising the cross-linked compound bound to the biological target; and
(e) obtaining the sequence of the unique DNA barcode of each of the one or more complexes comprising the cross-linked compound bound to the biological target,
wherein the sequence of the unique DNA barcode identifies the compound bound to the biological target.
